SEQUENCE LISTING (14 sequences)

SEQ ID NO: 1 - protein, 354 aa, Homo sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDVAVQACLNDGAQIAKVGQIFAAWKILGYDRCDAGWLADGSVRYPISRPRRRCSPTEAAVRFVGFPDKKHKLYGVYCFRAYN

SEQ ID NO: 2 - DNA, 1065 bp, Homo sapiens
atgaagagtctacttcttctggtgctgatttcaatctgctgggctgatcatctttcagacaactatactctggatcatgacagagctattcacatccaagcagaaaatggcccccatctacttgtggaagcagagcaagccaaggtgttttcacacagaggtggcaatgttacactgccatgtaaattttatcgagaccctacagcatttggctcaggaatccataaaatccgaattaagtggaccaagctaacttcggattacctcaaggaagtggatgtttttgtttccatgggataccacaaaaaaacctatggaggctaccagggtagagtgtttctgaagggaggcagtgatagtgatgcttctctggtcatcacagacctcactctggaagattatgggagatataagtgtgaggtgattgaaggattagaagatgatactgttgtggtagcactggacttacaaggtgtggtattcccttactttccacgactggggcgctacaatctcaattttcacgaggcgcagcaggcgtgtctggaccaggatgctgtgatcgcctccttcgaccagctgtacgacgcctggcggggcgggctggactggtgcaatgccggctggctcagtgatggctctgtgcaatatcccatcacaaagcccagagagccctgtggggggcagaacacagtgcccggagtcaggaactacggattttgggataaagataaaagcagatatgatgttttctgttttacatccaatttcaatggccgtttttactatctgatccaccccaccaaactgacctatgatgaagcggtgcaagcttgtctcaatgatggtgctcagattgcaaaagtgggccagatatttgctgcctggaaaattctcggatatgaccgctgtgatgcgggctggttggcggatggcagcgtccgctaccccatctctaggccaagaaggcgctgcagtcctactgaggctgcagtgcgcttcgtgggtttcccagataaaaagcataagctgtatggtgtctactgcttcagagcatacaactga

SEQ ID NO: 3 - DNA, 23 bp, Artificial Sequence
  primer
gtgcttattggttctccgttagt

SEQ ID NO: 4 - DNA, 21 bp, Artificial Sequence
  primer
cacaagccagaaatcctccat

SEQ ID NO: 5 - DNA, 21 bp, Artificial Sequence
  primer
gcctgtggagtacaagtcctt

SEQ ID NO: 6 - DNA, 23 bp, Artificial Sequence
  primer
aattcgggtgaagttatctgtgg

SEQ ID NO: 7 - DNA, 17 bp, Artificial Sequence
  primer
gccgcgcggaaaagatg

SEQ ID NO: 8 - DNA, 20 bp, Artificial Sequence
  primer
acatcgtgcagaagtaggca

SEQ ID NO: 9 - DNA, 19 bp, Artificial Sequence
  primer
tctcggtgtgatctgaggg

SEQ ID NO: 10 - DNA, 19 bp, Artificial Sequence
  primer
tggcgaacctgtagctgga

SEQ ID NO: 11 - DNA, 20 bp, Artificial Sequence
  primer
aatgcctttgatggaccaat

SEQ ID NO: 12 - DNA, 20 bp, Artificial Sequence
  primer
tagggttgctggggtagatg

SEQ ID NO: 13 - DNA, 21 bp, Artificial Sequence
  primer
tcacacaaaggaccagaatcg

SEQ ID NO: 14 - DNA, 24 bp, Artificial Sequence
  primer
tggtaatcttgaagtctcgaaagg

(19) United States
(12) Patent Application Publication
(10) Pub. No.: US 2022/0016203 A1
(43) Pub. Date: Jan. 20, 2022

(54) COMPOSITIONS AND METHODS FOR PREVENTION AND REDUCTION OF METASTASIS

(71) Applicant: The Wistar Institute of Anatomy and Biology, Philadelphia, PA (US)

(72) Inventor: Ashani T. Weeraratna, Merion Station, PA (US)

(21) Appl. No.: 17/281,796

(22) PCT Filed: Oct. 2, 2019
(86) PCT No.: PCT/US19/54288

§ 371 Date: Mar. 31, 2021

Related U.S. Application Data
(60) Provisional application No. 62/740,330, filed on Oct. 2, 2018.

(51) Int. Cl. A61K 38/17; A61K 48/00; A61P 35/04; C12N 15/86; G01N 33/68; G01N 33/574

(52) U.S. Cl. CPC A61K 38/1709; A61K 48/005; A61P 35/04; C12N 2740/15032; G01N 33/6887; G01N 33/5743; C12N 2740/15043; C12N 15/86

(57) ABSTRACT

Compositions and methods for the prevention or reduction of metastasis are provided. Such compositions and methods include increasing the level or expression of HAPLN 1.

### STATEMENT OF GOVERNMENT SUPPORT

This invention was made with government support under contract nos. NIH R01CA174746, R01CA207935, P50 CA174523, K99 CA208012-01 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “WST177PCT_Sequence-Listing_ST25.txt”.

### BACKGROUND OF THE INVENTION

The progression of cancer to distant metastatic sites is the single most prognostic factor for many solid organ malignancies (1, 2). In the specific case of cutaneous melanoma, increasing age identifies a high-risk group (3), with more frequent development of incurable visceral metastasis (4). The prognostic role of age, and its impact on the clinical course of melanoma, is unlikely to be fully explained by differences in the primary tumor, as elderly patients demonstrate shorter disease-specific survival even when adjusting for known pathologic variables (3, 4). The biological effects of aging on patterns of tumor dissemination have been largely unexplored. Whether age related changes in tumor phenotype drive lymphatic versus hematogenous metastasis has important clinical implications, as regional lymphatic metastases are frequently treated with surgical resection, whereas distant metastases are often incurable. Clinical observation supports initial melanoma spread through the intradermal lymphatics to regional nodal basins, with subsequent progression to distant sites (5). Tumor invasion into the lymphatic conduit allows passage to regional lymph nodes, which are secondary immune organs that function to filter lymph by containing antigens and providing a context for antigen presentation to immune cells (6). In the absence of distant metastases, nodal involvement is the most prognostic factor for survival. Current National Comprehensive Cancer Network (NCCN) guidelines support the use of sentinel node biopsy (SNB) to evaluate the tumor status of the regional lymphatic drainage basin in patients with clinically localized melanoma. However, recent data have shown that lymph node dissection did not increase melanoma-specific survival among melanoma patients (7). Notably, older patients demonstrate a lower incidence of SNB metastases—which should confer a more favorable prognosis—yet paradoxically have inferior disease-specific survival compared with younger patients (8, 9), which cannot be simply explained by age related changes in the false negative results of SNB (9). No biologic mechanism has yet been identified that accounts for this clinical observation.

It has previously been demonstrated that changes in the aging tumor microenvironment, such as secreted factors from dermal fibroblasts, can promote melanoma invasion and may account for inferior age-dependent clinical outcomes. In our analysis of the young and aged fibroblast secretome, the most highly secreted protein by young fibroblasts is the hyaluronan and proteoglycan link protein 1 (HAPLN1) (10), which crosslinks hyaluronan to the extracellular matrix (ECM). Hyaluronan alters the ability of fibroblasts to contract collagen matrices(11) and its role in cancer is believed to be tissue specific; reduced hyaluronan is associated with increased tumorigenesis in normally hyaluronan-rich tissues such as skin(12). Our recent data show that HAPLN1 loss in the aged tumor microenvironment drives a breakdown in the crosslinking of the ECM, which promotes melanoma migration while limiting intravasation of tumor-infiltrating lymphocytes (13).

### SUMMARY OF THE INVENTION

Provided herein are methods and compositions related to HAPLN1 and its role in cancer progression and metastasis. In one aspect, a method of preventing, inhibiting or decreasing cancer metastasis is provided. The method includes upregulating or delivering HAPLN1 to a subject in need thereof.

In another aspect, a method of decreasing lymphatic vessel permeability is provided. The method includes upregulating or delivering HAPLN1 to a subject in need thereof.

In yet another aspect, a method of decreasing, inhibiting or preventing visceral metastasis is provided. The method includes upregulating or delivering HAPLN1 to a subject in need thereof.

In another aspect, a method of predicting the likelihood of survival for a subject having cancer is provided. The method includes assaying for HAPLN1 protein or RNA expression in lymphatic tissue. In one embodiment, a higher HAPLN1 level is indicative of a higher chance of survival.

In another aspect, a method of predicting likelihood of metastasis in a subject is provided. The method includes assaying for HAPLN1 expression in lymphatic tissue. In one embodiment, a higher HAPLN1 level is indicative of a lower chance of metastasis.

In another aspect, a method of preventing ovarian cancer or ovarian cancer metastasis in a subject is provided. The method includes upregulating or delivering HAPLN1 to the ovaries.

Other aspects and advantages of the invention will be readily apparent from the following detailed description of the invention.

### BRIEF DESCRIPTION OF THE DRAWINGS

FIG. 1A-FIG. 1F demonstrate that retention of technicium dye and patterns of metastatic dissemination correlate with age. (FIG. 1A) Consecutive melanoma patients (n=1081) were injected with ^{99m}Tc sulfur colloid in the region of the primary tumor and a gamma probe was used to quantify the signal intensity in the surgically resected sentinel lymph node (Spearman's p=−0.30, p<0.001) (FIG. 1B) Representative lymphoscintigraphy of Tc-99m sulfur colloid signal in young and aged melanoma patients; (FIG. 1C) Survival curve adjusted for #of positive SLN, T-stage and ulceration, stratified by hottest counts above or below the median (HR 0.831, p=0.0014 95% CI 0.719-0.92); (FIG. 1D) Kaplan-Meier analysis of distant metastatic-free survival in sentinel lymph node biopsy negative patients, stratified by patient age at diagnosis (n=1,649; log rank p<0.001); (FIG. 1E) mCherry-labeled Yumm1.7 cells were injected into young or aged C57BL/6 mice and metastatic cells were identified in the draining inguinal lymph node by immunohistochemistry (bar=100 μm). Number of cells were counted per lymph node and graphed (two-tailed unpaired t-test, p=0.0030); (FIG. 1F) The mean number of metastases per high power field was similarly determined for lungs from the matching young and aged C57BL/6 mouse cohort (two-tailed unpaired t-test, p=0.0029).

FIG. 2A-FIG. 2H demonstrate changes in lymphatic fibroblast ECM deposition according to age. (FIG. 2A) GSEA analysis for ECM fibril organization of lymph nodes from human melanoma patients (n=221; nominal p=0.008); (FIG. 2B) In vitro extracellular matrix was produced by lymphatic fibroblasts isolated from young and aged donors and analyzed for the levels of fiber orientation by fibronectin immunofluorescence. The fiber distribution was determined by calculating the percent of fibers arranged in parallel for each acquired region (±90° of the mode angle); each point in the corresponding dotplot represent the mean number of fibers orientated at each angle (paired bars represent the standard error of the mean); (FIG. 2C) Analysis of extracellular matrix orientation produced in vitro by dermal fibroblasts from young or aged healthy donors; (FIG. 2D) Schematic of experimental setup: Matrices derived from young and aged fibroblasts were reconstituted with HUVECs followed by incubation with Texas red dye in the upper transwell chamber. Permeability was determined by quantification of the fluorescence of the lower chamber after 30 minutes. (FIG. 2E) Permeability of endothelial cells plated on young and aged matrices as measured by Texas red (two-tailed unpaired t-test: young vs. aged, p<0.001); (FIG. 2F) Transwell permeability assay of HUVECs plated on acellular extracellular matrices produced by extracted aged fibroblasts treated with increasing doses of rHAPLN1 (ANOVA p=0.0032; two-tailed unpaired t-test: 0 ng vs. 5 ng, p=0.0205; 0 ng vs. 25 ng, p=0.0080); (FIG. 2G) Transwell permeability assay of HUVECs plated on acellular extracellular matrices produced by extracted young fibroblasts with shHAPLN1 knockdown (ANOVA p=0.0004; two-tailed unpaired t-test: shempty vs. sh0501, p=0.0004; shempty vs. sh3400, p=0.0056); (FIG. 2H) Quantification of GFP-labeled melanoma cell migration (48 hours) on cell derived matrices under different conditions (ANOVA p=0.0004; all pairwise comparisons by two-tailed t-tests, p<0.001).

FIG. 3A-FIG. 3F demonstrate changes in cell-adhesion and integrin expression with age. (FIG. 3A) Representative VE-cadherin (middle) confocal immunofluorescence of HUVECs plated on acellular matrices following extraction of young shEmpty and shHAPLN1 fibroblasts and aged fibroblasts treated with rHAPLN1 (25 ng/mL) or PBS (bar=100 μm); (FIG. 3B) Corresponding quantification of the signal intensity of VE-cadherin positive cellular adhesions between HUVECs; (FIG. 3C) Relative expression by qtPCR of integrins (ITGB1, ITGB5, IGTA1, ITGA5) and CD44 of HUVECs on acellular matrices produced by young or aged fibroblasts (** p<0.01); (FIG. 3D) Relative expression by qtPCR of integrins (ITGB1, ITGB5, IGTA1, ITGA5) and CD44 of HUVECs on acellular matrices produced by young fibroblasts following HAPLN1 knockdown (* p<0.05; ** p<0.01); (FIG. 3E) Representative podoplanin (brown) and VE-cadherin (red) two-color immunohistochemistry of human sentinel lymph node specimens from primary cutaneous melanoma patients (n=16; bar=100 μm); (FIG. 3F) VE-cadherin signal quantification from human sentinel lymph nodes (n=16; two-tailed unpaired t-test, p=0.0003).

FIG. 4A-FIG. 4I demonstrate effects of HAPLN1 on lymph node integrity. (FIG. 4A) Representative two-photon microscopy of pericapsular collagen structure of inguinal lymph node in young or aged C57BL/6 mice, and corresponding quantification; (FIG. 4B) HAPLN1 mRNA expression as measured by RT-PCR. (FIG. 4C) Representative two-photon microscopy of pericapsular collagen structure of inguinal lymph node in aged C57BL/6 mice treated with rHAPLN1 (100 ng twice weekly) or PBS; (FIG. 4D) Quantification of young and aged lymphatic pericapsular extracellular matrix fiber orientation by collagen fluorescence. Each point in the corresponding dotplot represent the mean number of fibers orientated at each angle (paired bars represent the standard error of the mean). The fiber distribution was determined by calculating the percent of fibers arranged in parallel for each acquired region (±90° of the mode angle); the percent of fibers within 15° of the mode was compared between study arms (two-tailed unpaired t-test, p=0.0009); (FIG. 4E) Quantification of lymphatic pericapsular extracellular matrix fiber orientation by collagen fluorescence after HAPLN1 treatment; (FIG. 4F) Representative HAPLN1 immunohistochemistry of sentinel lymph node specimens of clinically node-negative melanoma patients (bar=100 μm). Each sample was assigned a H-score that included the relative signal intensity and area of staining (n=30; two-tailed unpaired t-test, p=0.0310); (FIG. 4G) Age-stratified TCGA analysis of HAPLN1 mRNA expression in regional lymphatic tissue of primary melanoma patients (n=192; two-tailed unpaired t-test, p=0.0324); (FIG. 4H) Geiger counts of melanoma patients following sentinel lymph node biopsy with Tc-99m sulfur colloid injection, stratified by HAPLN1-positivity by immunohistochemistry staining (n=86; two-tailed unpaired t-test, p=0.0046); (FIG. 4I) Kaplan-Meier survival function of non-metastatic human melanoma patients in the TCGA database, stratified by quartiles of regional lymph node HAPLN1 mRNA (n=192; log rank p<0.001).

FIG. 5A-FIG. 5E demonstrate in vivo effects of HAPLN1 on routes of metastatic dissemination. (FIG. 5A) mCherry-labeled yumm 1.7 cells were injected into aged C57BL/6 mice (n=18/arm) and the draining lymph nodes were treated with HAPLN1 (100 ng) or PBS. Tumor metastasis in the draining lymphatics were identified by immunohistochemical staining (chi squared p=0.0237); (FIG. 5B) Lymphatic tumor burden was quantified (two-tailed unpaired t-test, p=0.0308); (FIG. 5C) Representative immunohistochemistry of mCherry-positive metastasis (red) in draining lymphatics; (FIG. 5D) Tumor metastasis in the lung in the identical mouse cohort were identified by immunohistochemical staining (chi squared p=0.0087); (FIG. 5E) Representative immunohistochemistry of mCherry-positive metastasis (red) in the lungs.

FIG. 6 is a schematic representation of age-dependent changes in melanoma tumor progression. Age-related changes in the peri-lymphatic stroma impair the integrity of lymphatic vessels and nodes and increase lymphatic permeability. Such differences may underlie the clinical observations of increased rates of in-transit disease and false negative sentinel lymph node biopsies.

FIG. 7A-FIG. 7D demonstrate that HAPLN1 Mediates Extracellular Matrix Complexity. (FIG. 7A) Analysis of extracellular matrix orientation produced in vitro by aged fibroblasts treated with increasing levels of rHAPLN1. Fibronectin fiber distribution was determined by calculating the percent of fibers arranged in parallel for each acquired region (±90° of the mode angle); each point in the corresponding dotplot represent the mean number of fibers orientated at each angle (paired bars represent the standard error of the mean); (FIG. 7B) Transwell permeability assay of HUVECs plated on acellular extracellular matrices produced by extracted aged fibroblasts treated with increasing doses of rHAPLN1. *=p<0.05. (FIG. 7C) HAPLN1 knockdown from young fibroblast line, demonstrated by qtPCR (two-tailed unpaired t-test, p=0.0083); (FIG. 7D) Analysis of extracellular matrix orientation produced in vitro by a young fibroblast with shHAPLN1 knockdown and empty vector control.

FIG. 8A-FIG. 8D show endothelial VE-cadherin expression following age-related HAPLN1 manipulation. (FIG. 8A) Representative VE-cadherin confocal immunofluorescence of HUVECs plated on acellular matrices following extraction of varying cell lines of young and (FIG. 8B) aged fibroblasts; (FIG. 8C) Representative VE-cadherin confocal immunofluorescence of HUVECs plated on acellular matrices following extraction of varying young fibroblasts following shHAPLN1 knockdown or (FIG. 8D), varying aged fibroblasts following treatment with rHAPLN1 (25 ng/mL).

FIG. 9A-FIG. 9B show that murine lymphatic HAPLN1 varies by age and mediates VE-cadherin expression. (FIG. 9A) Representative fibronectin immunofluorescence of inguinal lymph nodes of young or aged C57BL/6 mice (bar=100 μm), and corresponding signal quantification in the pericapsular space (n=6/arm; two-tailed unpaired t-test, p=0.0023); (FIG. 9B) Representative LYVE-1 and VE-cadherin immunofluorescence of aged murine lymph nodes following treatment with rHAPLN1 (100 ng) or PBS control (bar=25 μm).

FIG. 10 is a graph showing the association between lymphatic HAPLN1 expression and overall survival. Kaplan-Meier survival function of non-metastatic human melanoma patients in the TCGA database, stratified by quartiles of regional lymph node HAPLN1 mRNA (n=192, log rank p=0.003).

FIG. 11A-FIG. 11C show primary tumors following lymphatic HAPLN1 treatment. (FIG. 11A) mCherry-labeled yumm 1.7 cells were injected into aged C57BL/6 mice (n=18/arm) and the draining lymph nodes were treated with HAPLN1 (100 ng) or PBS. Analysis of variance of tumor size was not significantly different between treatments (p=0.9337); (FIG. 11B) Representative CD31 immunohistochemistry of primary tumors from aged C57BL/6 mice following lymphatic treatment with HAPLN1 (100 ng) or PBS (bar=100 μm); (FIG. 11C) Quantification of CD31-positive vessels (n=8/arm; two-tailed unpaired t-test, p=0.3558).

### DETAILED DESCRIPTION OF THE INVENTION

The compositions and methods described herein are useful in the prevention or reduction in the risk of developing cancer metastasis. Older melanoma patients have lower rates of sentinel lymph node (LN) metastases yet paradoxically have inferior survival. Patient age correlated with an inability to retain Technetium radiotracer during sentinel LN biopsy in over 1000 patients, and high technecium counts correlated to better survival.

HAPLN1 has a key role in the age-dependent regulation of the extracellular matrix (ECM). Lymphatic HAPLN1 expression was prognostic of long-term patient survival in a multivariate Cox proportional hazards model adjusting for disease stage and patient age. The addition of rHAPLN1 to aged fibroblast ECMs in vitro was sufficient to reduce endothelial permeability via modulation of VE-Cadherin junctions, whereas endothelial permeability was increased following HAPLN1 knockdown in young fibroblasts. In vivo, reconstitution of HAPLN1 in aged mice increased the number of lymph node metastases, while simultaneously reducing the visceral metastases.

The studies described herein reveal that changes in the stroma during aging may influence the way tumor cells traffic through the lymphatic vasculature. Aging dictates the route of metastatic dissemination of tumor cells, and understanding these changes can reveal targetable moieties in the aging tumor microenvironment.

Human skin is characterized by an epidermal layer comprised primarily of keratinocytes and a dermal layer comprising mostly of dense collagen-rich extracellular matrix (ECM) largely secreted by dermal fibroblasts. Hyaluronan and proteoglycan link protein 1 (HAPLN1) is an ECM protein, highly expressed in young fibroblasts. Genetic polymorphisms of HAPLN1 have been associated with intervertebral disc degeneration (Mayer et al, Spine J. 2013 March; 13(3):299-317), and HAPLN1 has been shown to be overexpressed in metastatic melanoma and secreted by the tumor cells (Naba et al, Molecular and Cellular Proteomics, 2012 April; 11(4):M111.014647. Epub 2011 Dec. 9, which is incorporated herein by reference). The sequence of human HAPLN1 is known and can be found at GenBank Accession No: AAH57808.1, Gene ID: 1404.

| human HAPLN1 | |
|---|---|
| SEQ ID NO: 1 | |
| 1 | mksllllvli sicwadhlsd nytldhdrai hiqaengphl lveaeqakvf shrggnvtlp |
| 61 | ckfyrdptaf gsgihkirik wtkltsdylk evdvfvsmgy hkktyggyqg rvflkggsds |
| 121 | daslvitdlt ledygrykce viegleddtv vvaldlqgvv fpyfprlgry nlnfheaqqa |
| 181 | cldqdavias fdqlydawrg gldwcnagwl sdgsvqypit kprepcggqn tvpgvrnygf |
| 241 | wdkdksrydv fcftsnfngr fyylihptkl tydvavqacl ndgaqiakvg qifaawkilg |
| 301 | ydrcdagwla dgsvrypisr prrrcsptea avrfvgfpdk khklygvycf rayn |

As used herein, HAPLN1 includes HAPLN1 and homologs from all sources, including human. The source of the HAPLN1 may be human, or another mammal such as non-human primate, bovine, ovine, porcine, caprine, or murine. The term includes human HAPLN1 of SEQ ID NO: 1, as well as all isoforms, analogs, functional fragments (polypeptides), functional derivatives, and functional variants thereof. See, Uniprot entry for HAPLN1, including, without limitation, entries D6RBS1, D6RFI7, D6RG04, D6RBX9, D6RC59, and D6RAK7, all of which are incorporated herein by reference. HAPLN1 also includes sequences sharing at least 90%, at least 95%, at least 97%, and at least 99% identity with SEQ ID NO: 1 or the other sequences described herein. HAPLN1 also includes both the full-length protein (including signal peptide), i.e., amino acids 1-354, as well as the mature protein, i.e., amino acids 16-354. Functional fragments include aa 1-199, 16-199, 1-153, 16-153, 1-258, 16-258, 1-169, and 16-169, all of SEQ ID NO: 1, as well as sequences sharing at least 90%, at least 95%, at least 97%, and at least 99% identity with those fragments.

As used herein, “treatment” refers to increasing the level, expression or activity of HAPLN1.

The terms “analog”, “modification” and “derivative” refer to biologically active derivatives of the reference molecule that retain desired activity as described herein. In general, the term “analog” refers to compounds having a native polypeptide sequence and structure with one or more amino acid additions, substitutions (generally conservative in nature) and/or deletions, relative to the native molecule, so long as the modifications do not destroy activity and which are “substantially homologous” to the reference molecule as defined herein. Preferably, the analog, modification or derivative has at least the same desired activity as the native molecule, although not necessarily at the same level. The terms also encompass purposeful mutations that are made to the reference molecule. Particularly preferred modifications include substitutions that are conservative in nature, i.e., those substitutions that take place within a family of amino acids that are related in their side chains. Specifically, amino acids are generally divided into four families: acidic, basic, non-polar and uncharged polar. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the molecule of interest may include up to about 5-20 conservative or non-conservative amino acid substitutions, so long as the desired function of the molecule remains intact. One of skill in the art can readily determine regions of the molecule of interest that can tolerate change by reference to Hopp/Woods and Kyte Doolittle plots, well known in the art.

By “fragment” is intended a molecule consisting of only a part of the intact full-length polypeptide sequence and structure. The fragment can include a C terminal deletion, an N terminal deletion, and/or an internal deletion of the native polypeptide. A fragment will generally include at least about 5-10 contiguous amino acid residues of the full length molecule, preferably at least about 15-25 contiguous amino acid residues of the full length molecule, and most preferably at least about 20 50 or more contiguous amino acid residues of the full length molecule, or any integer between 5 amino acids and the full length sequence, provided that the fragment in question retains the ability to elicit the desired biological response, although not necessarily at the same level.

The term “derived from” is used to identify the original source of a molecule (e.g., bovine or human) but is not meant to limit the method by which the molecule is made which can be, for example, by chemical synthesis or recombinant means.

By “vector” is meant an entity that delivers a heterologous molecule to cells, either for therapeutic or vaccine purposes. As used herein, a vector may include any genetic element including, without limitation, naked DNA, a phage, transposon, cosmid, episome, plasmid, or a virus. Vectors are generated using the techniques and sequences provided herein, in conjunction with techniques known to those of skill in the art. Such techniques include conventional cloning techniques of cDNA such as those described in texts such as Sambrook et al, Molecular Cloning: A Laboratory Manual, 3^{rd} edition, 2001 Cold Spring Harbor Press, Cold Spring Harbor, N.Y., and current editions thereof, use of overlapping oligonucleotide sequences of the adenovirus genomes, polymerase chain reaction, and any suitable method which provides the desired nucleotide sequence. Suitable vectors include viral vectors such as adenovirus, adeno-associated virus, retrovirus, and lentivirus, amongst others.

“Expression control sequences” include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized in the construction of the compositions and performance of the methods described herein.

“Patient” or “subject” as used herein means a mammalian animal, including a human male or female, a veterinary or farm animal, e.g., horses, livestock, cattle, pigs, etc., a domestic animal or pet, e.g., dogs, cats; and animals normally used for clinical research, such as primates, rabbits, and rodents. In one embodiment, the subject of these methods and compositions is a human. Further, the terms include those of all ages. In one embodiment, the subject is an older, or aged, adult. In one embodiment, an older adult is at least 50 years old. In one embodiment, an older adult is at least 55 years old. In one embodiment, an older adult is at least 60 years old. In another embodiment, an older adult is at least 65 years old. In one embodiment, an older adult is at least 70 years old. In one embodiment, an older adult is at least 75 years old. In yet another embodiment, an older adult is at least 80 years old. In some embodiments, subjects that may benefit from the diagnostic/predictive methods of the invention include aged adults and adults with negative SNB (potentially false negatives).

As used herein, the term “treatment of cancer” or “treating cancer” can be described by a number of different parameters including, but not limited to, reduction in the size of a tumor in an animal having cancer, reduction in the growth or proliferation of a tumor in an animal having cancer, preventing metastasis or reducing the extent of metastasis, and/or extending the survival of an animal having cancer compared to control. In certain embodiments, treatment results in a reduced risk of distant recurrence or metastasis, including visceral metastasis.

In one embodiment, the subject has cancer selected from melanoma, prostate, clear cell renal cell carcinoma, breast cancer, other skin cancers, and any other cancers that can metastasize via the lymphatic system, including but not limited to lung, non-small cell lung, pancreatic, colorectal, head and neck, cervical, endometrial, testicular, and ovarian cancer. In one embodiment, the cancer is melanoma.

As used herein, the terms “increased duration of survival” or “increased survival” refers to the propensity of a patient with a disease or condition to live longer than predicted compared to another patient diagnosed with the same disease or condition. Survival may be, for example, survival without progression of the disease or cancer or overall patient survival. In certain embodiments, increased survival refers to the time interval between date of diagnosis or first treatment (such as surgery or first chemotherapy) and a specified event, such as relapse, metastasis, or death. Overall survival is the time interval between the date of diagnosis or first treatment and date of death or date of last follow up. Relapse-free survival is the time interval between the date of diagnosis or first treatment and date of a diagnosed relapse (such as a locoregional recurrence) or date of last follow up. Metastasis-free survival is the time interval between the date of diagnosis or first treatment and the date of diagnosis of a metastasis or date of last follow up.

In some examples, treatment of cancer by altering the expression, level, or activity of HAPLN1 can include increasing survival, for example, overall survival, relapse-free survival, or metastasis-free survival, such as increased survival time compared to in the absence of treatment. Such increased survival can include e.g., survival time of at least about 6 months from time of diagnosis, such as about 12 months, such as about 20 months, such as about 30 months, such as about 40 months, such as about 50 months, such as about 60 months, about 80 months, about 100 months, about 120 months or about 150 months from time of diagnosis or first treatment.

In some embodiments, a subject is screened to determine if they would benefit from treatment with an agent that alters (increases or decreases) expression or activity of HAPLN1. In certain embodiments, expression of HAPLN1 is determined in a sample from the subject. If the expression of HAPLN1 is altered (for example increased or decreased) relative to a control sample, the subject may be treated with an agent that alters (increases or decreases) expression or activity of HAPLN1.

Compositions

Provided herein are HAPLN1 compositions and methods of utilizing same. HAPLN1 may be delivered as a recombinant form or provided via vector such that the protein is produced in vivo. Recombinant forms of HAPLN1 are available commercially (e.g., catalog no. 2608-HP-025 from R&D Systems) or may be produced recombinantly using techniques known in the art, using the native coding sequence (SEQ ID NO: 2) and degenerate coding sequences, including codon optimized sequences. See, Ho and Gibaldi, Ch. 5: Large-scale production of recombinant proteins, in Biotechnology and Biopharmaceuticals: Transforming Proteins and Genes into Drugs, October 2013, John Wiley & Sons.

| hHAPLN1 nucleic acid | |
|---|---|
| SEQ ID NO: 2 | |
| atgaagagtc tacttcttct ggtgctgatt tcaatctgct gggctgatca tctttcagac | 60 |
| aactatactc tggatcatga cagagctatt cacatccaag cagaaaatgg cccccatcta | 120 |
| cttgtggaag cagagcaagc caaggtgttt tcacacagag gtggcaatgt tacactgcca | 180 |
| tgtaaatttt atcgagaccc tacagcattt ggctcaggaa tccataaaat ccgaattaag | 240 |
| tggaccaagc taacttcgga ttacctcaag gaagtggatg tttttgtttc catgggatac | 300 |
| cacaaaaaaa cctatggagg ctaccagggt agagtgtttc tgaagggagg cagtgatagt | 360 |
| gatgcttctc tggtcatcac agacctcact ctggaagatt atgggagata taagtgtgag | 420 |
| gtgattgaag gattagaaga tgatactgtt gtggtagcac tggacttaca aggtgtggta | 480 |
| ttcccttact ttccacgact ggggcgctac aatctcaatt ttcacgaggc gcagcaggcg | 540 |
| tgtctggacc aggatgctgt gatcgcctcc ttcgaccagc tgtacgacgc ctggcggggc | 600 |
| gggctggact ggtgcaatgc cggctggctc agtgatggct ctgtgcaata tcccatcaca | 660 |
| aagcccagag agccctgtgg ggggcagaac acagtgcccg gagtcaggaa ctacggattt | 720 |
| tgggataaag ataaaagcag atatgatgtt ttctgtttta catccaattt caatggccgt | 780 |
| ttttactatc tgatccaccc caccaaactg acctatgatg aagcggtgca agcttgtctc | 840 |
| aatgatggtg ctcagattgc aaaagtgggc cagatatttg ctgcctggaa aattctcgga | 900 |
| tatgaccgct gtgatgcggg ctggttggcg gatggcagcg tccgctaccc catctctagg | 960 |
| ccaagaaggc gctgcagtcc tactgaggct gcagtgcgct tcgtgggttt cccagataaa | 1020 |
| aagcataagc tgtatggtgt ctactgcttc agagcataca actga | 1065 |

HAPLN1-encoding nucleic acids may be used for a variety of purposes in accordance with the present invention. In a preferred embodiment of the invention, a nucleic acid delivery vehicle (i.e., an expression vector) for modulating lymphatic permeability or tumor metastasis is provided wherein the expression vector comprises a nucleic acid sequence coding for a HAPLN1 polypeptide, or a functional fragment thereof as described herein. Administration of HAPLN1-encoding expression vectors to a patient results in the expression of HAPLN1 polypeptide which alters lymphatic vessel permeability or tumor metastasis. In accordance with the present invention, a HAPLN1-encoding nucleic acid sequence may encode a HAPLN1 polypeptide as described herein whose expression reduces or prevents visceral metastasis or metastasis from the lymph nodes.

Expression vectors comprising HAPLN1-encoding nucleic acid sequences may be administered alone, or in combination with other molecules useful in preventing or treating cancer. According to the present invention, the expression vectors or combination of therapeutic agents may be administered to the patient alone or in a pharmaceutically acceptable or biologically compatible compositions.

In certain embodiments of the invention, the expression vector comprising nucleic acid sequences encoding the HAPLN1 is a viral vector. Viral vectors which may be used in the present invention include, but are not limited to, adenoviral vectors (with or without tissue specific promoters/enhancers), adeno-associated virus (AAV) vectors of multiple serotypes (e.g., AAV-2, AAV-5, AAV-7, and AAV-8) and hybrid AAV vectors, lentivirus vectors and pseudo-typed lentivirus vectors [e.g., Ebola virus, vesicular stomatitis virus (VSV), and feline immunodeficiency virus (FIV)], herpes simplex virus vectors, vaccinia virus vectors, and retroviral vectors.

HAPLN1 may be provided to the subject in need thereof in the form of a vector, which includes a coding sequence for HAPLN1 under appropriate expression control sequences to allow for expression of the protein in vivo. In certain embodiments, methods are provided for the administration of a viral vector comprising nucleic acid sequences encoding HAPLN1, or a functional fragment thereof. Exemplary vectors include adenoviral vectors. Such vectors preferably include at least the essential parts of adenoviral vector DNA. As described herein, expression of a HAPLN1 polypeptide following administration of such an adenoviral vector serves to, for example, decrease lymphatic vessel permeability or prevent or reduce tumor metastasis.

Due to their large size (about 36 kilobases), adenoviral genomes are well suited for use as gene therapy vehicles because they can accommodate the insertion of foreign DNA following the removal of adenoviral genes essential for replication and nonessential regions. Such substitutions render the viral vector impaired with regard to replicative functions and infectivity. Of note, adenoviruses have been used as vectors for gene therapy and for expression of heterologous genes. For a more detailed discussion of the use of adenovirus vectors utilized for gene therapy, see Berkner, 1988, Biotechniques 6:616-629 and Trapnell, 1993, Advanced Drug Delivery Reviews 12:185-199.

For some applications, an expression construct may further comprise regulatory elements which serve to drive expression in a particular cell or tissue type. Such regulatory elements are known to those of skill in the art and discussed in depth in Sambrook et al. (1989) and Ausubel et al. (1992) and include, without limitation, promoters, enhancers, polyA sequences, kozak sequences, etc. The incorporation of tissue specific regulatory elements in the expression constructs of the present invention provides for at least partial tissue tropism for the expression of the HAPLN1 or functional fragments thereof.

Pharmaceutical compositions may be in the form of liquid solutions or suspensions (as, for example, for intravenous administration, for oral administration, etc.). Alternatively, pharmaceutical compositions may be in solid form (e.g., in the form of tablets or capsules, for example for oral administration). In some embodiments, pharmaceutical compositions may be in the form of powders, drops, aerosols, etc.

Methods and agents well known in the art for making formulations are described, for example, in “Remington's Pharmaceutical Sciences,” Mack Publishing Company, Easton, Pa. Formulations may, for example, contain excipients, diluents such as sterile water, or saline, polyalkylene glycols such as polyethylene glycol, oils of vegetable origin, or hydrogenated napthalenes.

Methods

In one aspect, a method of decreasing lymphatic vessel permeability is provided. The method includes increasing the expression or level of HAPLN1 to a subject in need thereof. Lymphatic vessels, which drain from the tumor to the regional lymph nodes, reside upon a scaffold of extracellular matrix (ECM) secreted by fibroblasts, and are made up of fibrillar collagen (9). It has been shown that during aging lymphatic vessels are susceptible to permeability and degradation, affecting lymphatic transport (10). It is shown herein that age-dependent loss of lymphatic endothelial integrity functionally impairs the lymphatic system's capacity to contain tumor cells, allowing them to escape from lymph nodes to distant sites. Age-related changes in ECM, similar to that in the skin might act to affect lymphatic vessel permeability. Described herein is the novel role of the HAPLN1 loss in the aging extracellular matrix in mediating lymphatic endothelial permeability, thus permitting melanoma cells to escape from the lymphatic system to distant metastatic sites. In vivo lymphatic vessel permeability can be determined using sentinel node biopsy (SNB), as described in the Examples below. After treatment with HAPLN1, SNB may be performed and compared versus a baseline SNB done prior to treatment to determine whether vessel permeability is improved. As used herein, a “decrease” in vessel permeability may be compared to the vessel permeability (as determined by SNB) of the same subject at an earlier time. In another embodiment, a “decrease” refers to a decrease in vessel permeability as compared to a suitable control subject. Such control subjects include those of the same or similar age, gender, and/or disease state, as determined by the person of skill in the art.

In another aspect, a method of decreasing, inhibiting, or preventing metastasis is provided. The method includes increasing the level or expression of HAPLN1 to a subject in need thereof. As used herein, the term “metastasis” or “metastases” refers to the spread of a primary cancer to a secondary location including the lymph nodes (lymphatic metastases), blood and other organs (visceral metastases).

In another aspect, a method of decreasing, inhibiting, or preventing visceral metastasis is provided. The method includes upregulating or delivering HAPLN1 to a subject in need thereof. Melanoma patients of older age experience a higher rate of distant metastasis and inferior overall survival. In particular, the dissemination of melanoma cells beyond the primary site and regional lymphatic basin that are the primary targets for surgical extirpation, presents a clinical dilemma with poor therapeutic options. While the relationship between age and sentinel lymph node positivity has been previously described, herein described are (1) age-related alterations in the perilymphatic extracellular matrix that mediates lymphatic permeability via modulation of VE-Cadherin junctions, and (2) a novel role of HAPLN1 in lymphatic ECM integrity, including its prognostic role in human patients and its therapeutic value in reducing visceral metastases. Visceral metastases include metastases beyond the lymphatic system, i.e., to internal organs including the liver, lungs, and body cavities like the pleura and peritoneum.

In one embodiment, the therapeutic benefit is reduction or prevention of metastasis. A reduction in metastasis can be measured as compared to the statistical likelihood of occurrence of metastasis for a similar subject or control. The “similar subject” or control can be determined by the health care provider, depending on appropriate criteria. Such criteria include, amongst others, age, gender, type and/or stage of cancer who has not been subject to HAPLN1 treatment. A reduction or inhibition of metastasis can be measured relative to the incidence observed in the absence of the treatment and, in further testing, inhibits metastatic tumor growth. The tumor inhibition can be quantified using any convenient method of measurement. The incidence of metastasis can be assessed by examining relative dissemination (e.g., number of organ systems involved) and relative tumor burden in these sites. Metastatic growth can be ascertained by microscopic or macroscopic analysis, as appropriate. Tumor metastasis can be reduced by about 10%, 20%, 30%, 40%, 50%, 60%, 70%, 80%, 90%, 95% or greater.

Other therapeutic benefits or beneficial effects provided by the methods described herein may be objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumor or cancer, or metastasis. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore be a cure, such as destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

As discussed herein, HAPLN1 has been found in reduced levels in aged cancer patients, resulting in increased incidence of visceral metastasis. Thus, in one aspect, methods of increasing levels of HAPLN1 mRNA or protein are provided. In one embodiment, HAPLN1 levels are increased via the use of an agonist, such as human chorionic gonadotropin (hCG). Other methods include the use of expression activating oligonucleotides (WO2013173652, which is incorporated herein by reference). In another embodiment, HAPLN1 levels are increased via the use of a vector which expresses HAPLN1 in a host cell, as described herein. In yet another embodiment, recombinant HAPLN1 is delivered to the subject.

The methods include delivering a therapeutically effective amount of HAPLN1 or a vector encoding the same. The term “therapeutically effective amount” or “effective amount” refers to an amount of HAPLN1, HAPLN1 agonist or HAPLN1-expressing vector that when administered alone or in combination with an additional therapeutic agent to a cell, tissue, or subject is effective to prevent or ameliorate the tumor-associated disease condition or the progression of the disease, e.g., metastasis. A therapeutically effective dose further refers to that amount of the compound sufficient to result reduction, prevention or inhibition of metastasis. For example, when in vivo administration of recombinant HAPLN1 is employed, normal dosage amounts may vary from about 10 ng/kg to up to 100 mg/kg of subject body weight or more per dosage or per day, preferably about 1 μg/kg to 50 mg/kg, optionally about 100 μg/kg to 20 mg/kg, 500 μg/kg to 10 mg/kg, or 1 mg/kg to 10 mg/kg, depending upon the route of administration. Various routes of administration are useful in these methods. In one embodiment, the recombinant protein or vector is delivered to the tumor site itself. In another embodiment, the recombinant protein, agonist or vector is delivered to tumor draining lymph node (TDLN) or nodes or other lymph node or nodes. Draining lymph nodes refers to lymph notes that lie immediately downstream of tumors. In another embodiment, the recombinant protein, agonist or vector is delivered to an afferent lymph vessel.

Pharmaceutical compositions may be formulated for any appropriate route of administration. For example, compositions may be formulated for intravenous, parenteral, subcutaneous, intramuscular, intracranial, intraorbital, ophthalmic, intraventricular, intracapsular, intraspinal, intracisteral, intraperitoneal, intranasal, or aerosol administration. In some embodiments, pharmaceutical compositions are formulated for direct delivery to the tumor (intratumoral) or to the tumor environment. In another embodiment, pharmaceutical compositions are formulated for delivery to the lymph nodes.

Also provided herein is a method of predicting the likelihood of survival in a subject who has cancer. The method includes assaying for HAPLN1 protein or RNA expression in lymphatic tissue. In one embodiment, a higher HAPLN1 level as compared to a control is indicative of a higher chance of survival. Methods for assaying for HAPLN1 mRNA or protein levels are known in the art, and described herein. In one embodiment, a HAPLN1 mRNA or protein level in the upper quartile, as compared to a control population, is indicative of an increased chance of survival. In another embodiment, a higher than median level is indicative of an increased chance of survival. In another embodiment, subjects with high lymphatic HAPLN1 expression are 56% less likely to die, regardless of age and disease state.

Provided herein is a method of predicting likelihood of visceral metastasis in a subject who has cancer. The method includes assaying for HAPLN1 expression in lymphatic tissue. In one embodiment, a higher HAPLN1 level is indicative of a lower chance of visceral metastasis.

In another aspect, a method of preventing ovarian cancer or ovarian cancer metastasis in a subject is provided. In one embodiment, HAPLN1 is delivered to the ovaries. In a further embodiment, HAPLN1 is used to coat the ovaries to prevent localized dissemination, prevent ovarian cancer or ovarian cancer metastasis.

All scientific and technical terms used herein have their known and normal meaning to a person of skill in the fields of biology, biotechnology and molecular biology and by reference to published texts, which provide one skilled in the art with a general guide to many of the terms used in the present application. However, for clarity, certain terms are defined as provided herein.

The terms “a” or “an” refers to one or more, for example, “an assay” is understood to represent one or more assays. As such, the terms “a” (or “an”), “one or more,” and “at least one” are used interchangeably herein.

The term “about” as used herein when referring to a measurable value such as an amount, a temporal duration, and the like, is meant to encompass variations of up to ±10% from the specified value; as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.”

Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

The terms “compound”, “composition”, or “substance” as used herein may be used interchangeably to discuss the therapeutic composition.

### EXAMPLES

Age-dependent loss of lymphatic endothelial integrity may functionally impair the lymphatic system's capacity to contain tumor cells, allowing them to escape from lymph nodes to distant sites. The age-related changes in ECM, similar to those observed in skin might act to affect lymphatic vessel permeability. We have identified a novel role of HAPLN1 loss in the aging extracellular matrix in mediating lymphatic endothelial permeability, thus permitting melanoma cells to escape from the lymphatic system to distant metastatic sites. These findings have important implications for the surveillance and treatment of melanoma in an aging population.

The following example is provided for the purpose of illustration only and the invention should in no way be construed as being limited to this example but rather should be construed to encompass any and all variations that become evident as a result of the teaching provided herein.

### Example 1: Methods

### Cell Lines and Culture Conditions

Dermal fibroblast cell lines were obtained from Biobank at Coriell Institute for Medical Research. Human lymphatic fibroblasts were isolated from lymph nodes from young (<35 years) and aged (>50 years) human donors by ScienCell Research Laboratories (Carlsbad, Calif.) and characterized by their spindle-shaped morphology and fibronectin-positive staining. The fibroblasts were cultured in DMEM (Invitrogen) supplemented with 10% FCS and 4 mM L-Glutamine. HUVEC cells were obtained from Lonza and cultured in EGM-2MV media (CC-3202, Lonza). Yumm1.7 murine melanoma cells were cultured in DMEM supplemented with 10% FCS and 4 mM L-Glutamine. 1205lu melanoma cells were maintained in DMEM supplemented with 5% FCS and 4 mM L-Glutamine and cultured at 37° C. in 5% CO₂ and medium was replaced as required. Short tandem repeat profiling was done for melanoma cells and compared against our internal control of over 200 melanoma cell lines as well as control lines such as HeLa and 293T and the results are available upon request. Mycoplasma testing was carried out using a Lonza MycoAlert assay at the University of Pennsylvania Cell Center Services.

### Lentiviral Production and Infection

HAPLN1 shRNA was obtained from the TRC shRNA library available at The Wistar Institute (TRCN0000150501, TRCN0000153400). Sequencing based verification of all plasmids was performed at the Genomics facility at The Wistar Institute. Lentiviral production was performed according to the protocol suggested by the Broad Institute. Briefly, 293T cells are plated at 70% confluency and co-transfected with shRNA plasmid and the lentiviral packaging plasmids (pCMV-dR8.74psPAX2, pMD2.G). pLKO.1 empty vector was used as a control. For transduction, cells were treated with lentivirus overnight and allowed to recover for 24 hours before selection using puromycin (1 μg/ml).

### Lymphoscintography

Count data was collected as part of standard of care. The data were not collected as part of a prospective clinical trial. Patients provided written informed consent for the procedure. De-identified medical records were used to generate the analyses used in the paper and an exemption determination from IRB review was independently granted (FARM-SLNRETRO-0805). Sentinel lymph node biopsy was routinely performed perinstitutional standard protocols, using radiotracer dye with or with vital blue dye. Lymphoscintigraphy was typically performed after radiotracer dye injection at the site of the primary and a hand-held gamma probe was used intra-operatively to assist with identification of the lymph nodes with radiotracer uptake. More specifically, one mCi total of filtered Technetium-99m sulfur colloid was injected intradermally in 4 separate aliquots (0.1-0.2 mL each) surrounding the melanoma. A gamma camera with low energy parallel hole collimator was utilized to obtain static emission images of the injection site as well as transmission images of the sentinel node. To aid in the operative identification of the SLN, isosulfan blue dye (Lymphazurin; Tyco Healthcare, Norwalk, Conn., USA) (1%, 1-3 ml/case) was injected prior to skin incision. A hand-held gamma probe (C-Trak; Care Wise Medical Products Corporation, Morgan Hill, Calif., USA) was used to localize the sentinel node on the basis of accumulation of the Tc-99m sulfur colloid, where >10% of background counts was considered positive. The maximum counts of each lymph node were confirmed ex vivo following surgical removal.

### Immunohistochemistry

Patient samples were collected under IRB exemption approval (protocol EX21205258-1). FFPE sections were deparaffinized using xylene followed by rehydration through series of alcohol washes and finally PBS. Heat-mediated antigenretrieval was performed using citrate based retrieval buffer (Vector Labs, H-3300). Samples were blocked in peroxide blocking buffer (Thermo Scientific), followed by protein block (Thermo Scientific) and incubated in appropriate antibody at 4° C. overnight in a humidified chamber. Following day, samples were washed and incubated with a biotinylated secondary antibody (Thermo Scientific) followed by Streptavidin-HRP incubation. Samples were then washed in PBS and incubated in 3-amino-9-ethyl-1 carboazole (AEC) chromogen and counterstained with Mayer's hematoxylin, rinsed in dH2O and mounted in aquamount. For mouse samples to be incubated with mouse antibodies, samples were blocked for an additional hour with mouse on mouse block (MKB-2213, Vector Labs). Multiplexed IHC samples were developed initially with DAB chromogen (Thermo Scientific) followed by blocking with protein block and incubation with second primary antibody developed using AEC chromogen. Primary antibodies used were as follows, mCherry (1:500, NBP2-25157, Novus Biologicals), HAPLN1 (1:100, TA325115, Origene), podoplanin (1:100, 322M-14, Sigma Aldrich), VE-cadherin (1:50, MAB9381, R&D Systems), Lyve-1 (1:50, ab14917, abcam), CD31 (1:50, ab28364, abcam).

### Immunofluorescence

Samples were fixed in 4% paraformaldehyde for 20 minutes followed by 1 hour treatment with blocking buffer (0.2% each of triton-x, BSA, gelatin and casein and 0.02% Sodium azide). Cells were incubated in primary antibody and incubated overnight at 4° C. Following day, cells were washed in PBS and incubated with appropriate secondary antibodies (Alexa fluor series, Invitrogen, 1:2000) at room temperature for 1 hour. Cells were washed in PBS, incubated with DAPI (Invitrogen, 1:10,000) and mounted in Prolong Gold anti-fade reagent (Invitrogen). Images were captured on a Leica TCS SPII scanning laser confocal system. Primary antibodies used were as follows, Fibronectin (1:200, #F3648, Sigma Aldrich), VE-cadherin (Human: 1:50, MAB9381, R&D Systems; Mouse: 1:10, 138001, Biolegend), Lyve-1 (1:50, ab14917, abcam).

### Quantitative PCR

mRNA was harvested using phenol-chloroform method as described previously and cleaned using RNeasy mini kit (Qiagen). HUVECs were plated on fibroblast-derived matrices for 24 hours and harvested using Trizol. For the murine inguinal lymph nodes, they were harvested, freshly digested in Trizol, and homogenized (X). Samples were then mixed with chloroform and clear layer was collected and processed through RNA cleanup kit using manufacturer's protocol. RNA concentration was measured using Nanodrop 2000 (Thermo Scientific). One microgram of RNA was used to prepare cDNA using iscript cDNA synthesis kit (1708891, Bio-rad). cDNA was diluted 1:5 before use. Each 20 μl reaction comprised 1 μl Power SYBR Green Master Mix (4367659, Invitrogen), 1 μl primer mix (Final concentration 0.5 μM) and 1 μl cDNA. Standard curves were generated for each primer and used to perform relative quantification. All samples were normalized to 18S primer pair (AM1718, Invitrogen). Primer sequences were obtained from IDT (Coralville, Iowa) for

ITGA1
(SEQ ID NO: 3-forward-GTGCTTATTGGTTCTCCGTTAGT,
SEQ ID NO: 4-reverse-CACAAGCCAGAAATCCTCCAT)
ITGA5
(SEQ ID NO: 5-Forward-GCCTGTGGAGTACAAGTCCTT,
SEQ ID NO: 6-reverse-AATTCGGGTGAAGTTATCTGTGG),
ITGB1
(SEQ ID NO: 7-Forward-GCCGCGCGGAAAAGATG,
SEQ ID NO: 8-reverse-ACATCGTGCAGAAGTAGGCA),
ITGB5
(SEQ ID NO: 9-Forward-TCTCGGTGTGATCTGAGGG,
SEQ ID NO: 10-reverse-TGGCGAACCTGTAGCTGGA),
CD44
(SEQ ID NO: 11-Forward-AATGCCTTTGATGGACCAAT,
SEQ ID NO: 12-Reverse-TAGGGTTGCTGGGGTAGATG),
HAPLN1
(SEQ ID NO: 13-Forward-TCACACAAAGGACCAGAATCG,
SEQ ID NO: 14-Reverse-TGGTAATCTTGAAGTCTCGAAAGG).

### Preparation of Fibroblast Matrices

Fibroblast matrices were prepared as previously described(28). Briefly, in a 24 well plate, 12 mm coverslips (No. 1) were added and coated with 0.2% gelatin solution for 1 hour. Wells were washed with DPBS (without Ca2+ and Mg2+), followed by treatment with 1% glutaraldehyde for 30 minutes at room temperature. After washing with DPBS, coverslips were incubated with 1M ethanolamine for 30 minutes at room temperature. Coverslips were washed with DPBS and 1×105 fibroblasts were plated on the coverslips and incubated overnight at 37° C., 5% CO2. Following day, fresh media containing 50 μg/ml L-ascorbic acid was added to the wells. L-ascorbic acid was added daily to the wells with fresh media replacement every other day. rHAPLN1 (#2608-HP, R&D Systems) was added to the media at varied concentrations and replaced during media changes. Matrices were harvested after a total of 5 treatments and analyzed as described under various sections.

### Anisotropic Analysis of Fibroblast Matrices

Matrices were prepared using either dermal of lymphatic fibroblasts and fixed for 20 minutes in buffer containing 4% paraformaldehyde and 4 g/ml Sucrose. Matrices were stained for fibronectin as described above and imaged using Leica SP5 II Confocal System. Samples were imaged with 63× objective at 2× zoom power and each z-stack was 0.5 μM thick. Stacks were added until fibers were indiscernible and at least 9 measurements were taken for each sample. Images were analyzed using ImageJ Plugin OrientationJ (available for download at http://bigwww.epfl.ch/demo/orientation). Images were normalized for orientation using R and graphed. Source code for R used in these analyses has been previously published (13).

### 2-Photon Microscopy

Inguinal lymph nodes were collected from C57/BL6 mice, held in buffer solution under nylon mesh and imaged with a Leica TCS SP8 MP 2-photon intravital microscope (Leica Microsystems, Inc, Buffalo Grove, Ill.). The specific region of interest was the lymph node capsule. Collagen was visualized using second harmonic generation (SHG) from 900 nm excitation in a Chameleon XR Ti:Saphire laser (Coherent, Inc., Santa Clara, Calif.). SHG emission was captured in 12 bits, at 700 Hz, through a 25×/1.00 water immersion objective in reflected mode using a HyD detector with a standard DAPI filter set. Mouse tissue images shown are composites of 15 z-stacks with 10 mm step size. The images were further processed using Huygens Professional Deconvolution software (Scientific Volume Imaging, B.V., The Netherlands).

### Transwell Permeability Assay

Fibroblast matrices were prepared as described above in 24 well transwell plate (Costar, #3413). 0.2×105 fibroblasts were seeded and treated for 5 days with L-ascorbic acid. Following treatment, fibroblasts were lysed with extraction buffer (0.5% Triton X-100, 2 mM NH₄OH in DPBS) for 5 minutes at 37° C., 5% CO₂, followed by 1:1 dilution with DPBS and incubated overnight at 4° C. Next day, wells were washed and seeded with endothelial cells at 1×10⁵ cells per transwell and incubated at 37° C., 5% CO₂ for 36 hours. Media was collected from the wells, followed by 2 washes with DPBS. Next, 200 μl of 2 mg/ml dextran red (#R9379, Sigma Aldrich) solution was prepared in HBSS (without phenol red) containing 5% FCS in the upper chamber and 450 μl of 5% FCS in HBSS solution was added to the bottom chamber. 50 μl sample was collected from the wells at various time intervals and analyzed on EnVision multi-label microplate reader at The Wistar Institute Molecular Screening and Protein Expression Facility. Samples were normalized to blank and graphed.

### Melanoma Cell Migration in Organotypic Culture (Reconstruct)

Cultures were prepared using a modified approach as previously described(29). Cultures were prepared in a 4 well 35 mm glass bottom dish for optimal imaging (Greiner cellview #50590467, Thermofisher Scientific). An acellular bottom layer of collagen matrix (1.6 ml 10×EMEM [12-684F, Lonza], 0.16 ml L-glutamine, 1.82 ml heat inactivated FCS, 0.2 ml NaHCO₃[17-613E, Lonza], 14.8 ml Rat Tail Collagen I [final concentration 1.0 mg/ml, #354249, Corning] was added in the dish and allowed to solidify for 1 hour. Next, fibroblasts (6×104 cells) were harvested and mixed with 250 μl collagen matrix and allowed to set for 1 hour at 37° C. Next, HUVEC (1×105 cells) labeled with mCherry were added on the fibroblast layer and incubated for 48 hours at 37° C., 5% CO₂. Next, 1205lu melanoma cells labeled with GFP were plated at 1×10⁵ cells per well and incubated in media prepared with 1:1 ratio of EGM-2MV and DMEM 10% FCS. The following day, time-lapse images were acquired on a Leica TCS SP8 X WLL Scanning Confocal Microscope. Image deconvolution was performed using Huygens Professional and analyzed using NIS Elements Advanced software and graphed using Graphpad/Prism6.

### In Vivo Xenograft Assay

All animal experiments were approved by the Institutional Animal Care and Use Committee (IACUC) (IACUC #112503X_0) and were performed in an Association for the Assessment and Accreditation of Laboratory Animal Care (AAALAC) accredited facility. YUMM1.7 (1×105 cells) overexpressing mCherry were injected subcutaneously into aged (50 weeks) and young (8 weeks) C57/BL6 mice (#556, Charles River). Mice were treated as follows with rHAPLN1 (100 ng into the inguinal lymph node, #2608-HP, R&D Systems, twice weekly) or PBS as control, starting two weeks prior to tumor injection and continuing until sacrifice. Tumor sizes were measured every 3-4 days using digital calipers, and tumor volumes were calculated using the following formula: volume=0.5×(length×width2). Time-to-event (survival) was determined by a 5-fold increase in baseline volume (˜1000 mm3) and was limited by the development of skin necrosis. Mice were euthanized, lungs and lymph nodes were harvested and metastases counted. Half of the tissue was embedded in paraffin and half in optimal cutting temperature compound (O.C.T, Sakura, Japan City) and flash frozen for sectioning. Lungs and lymph nodes were sectioned and stained with mCherry (NBP2-25157, Novus Biologicals) to determine melanoma metastasis. All reagents injected in live mice were tested for endotoxin levels at University of Pennsylvania Cell Center Services using The Associates of Cape Cod LAL test.

### TCGA Database Analysis

The RNAseq and Clinical dataset for skin cutaneous melanoma (30) was downloaded from The Cancer Genome Atlas (TCGA; http://cancergenome.nih.gov/). Normalized mRNA expression was analyzed by quartiles. Patient ages were grouped into categories (<50, 51-79, and ≥80 years).

### Gene Set Expression Analysis

We performed a targeted analysis on GSEA gene signatures related to ECM organization. GO ECM fibril organization gene signature was downloaded from Molecular Signatures Database (http://software.broadinstitute.org/gsea/msigdb/index.jsp). GSEA analysis was performed using javaGSEA desktop application available from http://software.broadinstitute.org/gsea/downloads.jsp. TCGA samples that were processed from regional lymph node metastases were used in GSEA analysis. For these LN samples, the GSEA score matrix was organized as having samples in the column and signature genes in the rows. Any sample that did not include either patient age or did not have associated gene expression data for the sample was excluded in the analysis. Differentially enriched signatures were defined as having nominal p-value below 0.05 and FDR below 5%.

### Statistical Analyses

For in vitro studies, a Student's t test or Wilcoxon rank-sum test (Mann-Whitney) was performed for two-group comparison. Estimate of variance was performed, and parameters for the t test were adjusted accordingly using Welch's correction. ANOVA or Kruskal-Wallis test with post-hoc Bonferroni's or Holm-Sidak's adjusted P values was used for multiple comparisons. For in vivo studies, repeated measures ANOVA was calculated between samples. The Holm-Sidak correction was performed. For other experiments, Graphpad/Prism6 was used for plotting graphs and statistical analysis. Survival analyses included Kaplan-Meier log rank test and multivariable Cox regression for univariate and multivariate analyses, respectively. Data was represented as ±SEM. Significance was designated as follows: *, P<0.05; **, P<0.01; and ***, P<0.001.

### Example 2: Results

### Age Determines Patterns of Melanoma Dissemination

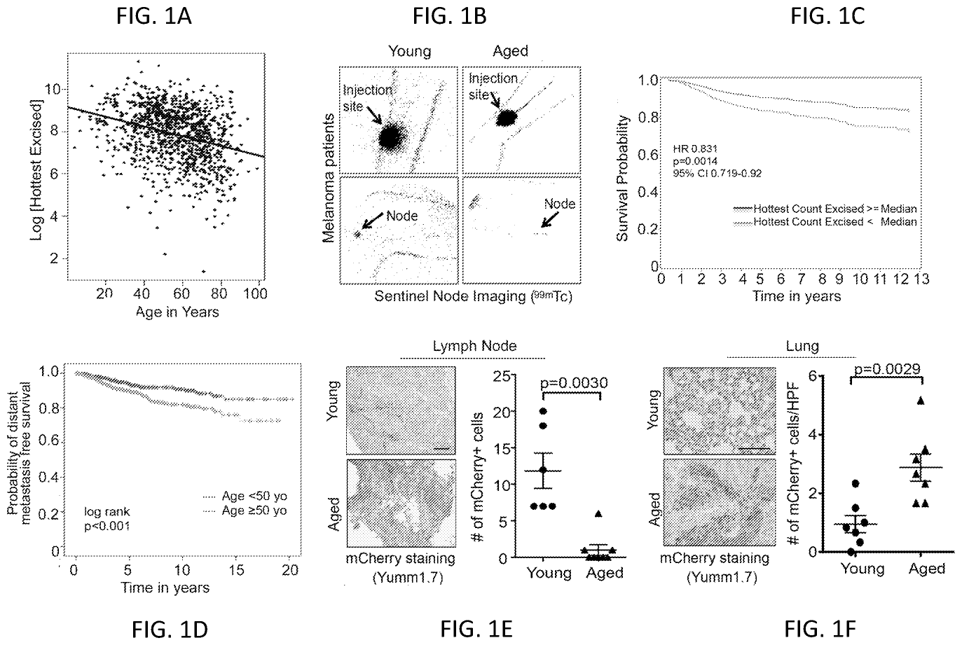

Consecutive American Joint Committee on Cancer [AJCC] Stage I-II melanoma patients underwent SNB using Technetium (^{99m}Tc) radiotracer (n=1081). SNB is a technique for the staging of subclinical regional metastatic disease using lymphatic mapping. 99mTc injected into the peritumoral skin is transported by dermal lymphatics on a direct pathway from the cutaneous site to the drainage lymph nodes (i.e., “sentinel” node). The sentinel node is subsequently identified in the operating room using a handheld gamma probe and surgically resected. There was a significant correlation with increasing age and lower ^{99m}Tc counts in the sentinel node (FIG. 1A, n=1081 patients, Spearman's ρ=−0.30, p<0.001), despite standardized injection protocols of radiotracer at the site of melanoma. Impaired retention of ^{99m}Tc was evident by decreased signal intensity in corresponding lymphoscintigraphy images (FIG. 1B). Patients provided written informed consent for the procedure. De-identified medical records were used to generate the analyses used in the paper and an exemption determination from IRB review was independently granted.

Decreased radiotracer counts may signify increased permeability through the lymphatic system and/or decreased transport to the draining lymph node. Aging is associated with decreases in the contraction frequency of lymphatic collectors, reducing lymph velocity (15, 16). Thus, patients with SNB which are negative for metastases may have (1) early stage disease that has not spread to the regional lymphatics (i.e., true negatives), (2) permeable lymphatics that allow for tumor migration through the SNB to reach the systemic circulation (i.e., false negative), or (3) tumor that cannot reach the lymph node due to faulty lymphatics. In accordance with our hypothesis that this loss of dye retention corresponded to both an inability to get to the lymph node (due to permeability issues) as well as an inability to be retained in the lymph node due to permeability of the lymph node capsule, rather than decreased rates of lymph node metastasis, survival (adjusted for number of positive lymph nodes, ulcerations and Tstage classification) was actually improved for patients who had higher than median levels of 99mTc in their lymph nodes (FIG. 1C, HR 0.831, p=0.0014, 95% CI 0.719-0.92). Multivariable analyses of these data in a multivariate Cox model, accounting for stage is shown as Table 1.

TABLE 1
Multivariable Cox Regression Model for overall survival
in 1081 patients (event = 131) undergoing SNB.

| Parameter | Comparison | HR* | 95% CI | P value |
|---|---|---|---|---|
| T-stage | T1 | Ref | Ref | — |
| | T2: 1.01-2.00 | 2.05 | 1.15-3.64 | 0.015 |
| | T3: 2.01-4.00 | 1.83 | 0.98-3.41 | 0.057 |
| | T4: >4 | 4.5 | 2.37-8.53 | <0.001 |
| | Unknown | 3.65 | 1.03-13.01 | 0.046 |
| Ulceration | No | Ref | Ref | — |
| | Yes | 1.9 | 1.29-2.82 | 0.001 |
| | Unknown | 0.5 | 0.12-2.18 | 0.358 |
| # positive SLN | As 1 unit increase | 2.31 | 1.86-2.87 | <0.001 |
| Log(hottest | As 1 log unit increase | 0.8 | 0.70-0.91 | 0.001 |
| excised) | | | | |

*Hazard ratio (HR) indicates relative hazard for death and was adjusted for all variables included

Given that aging appeared to be associated with increased SNB permeability, we hypothesized that older patients with negative SNB (i.e., no evidence of lymphatic metastases) would have a higher rate of distant, visceral metastasis. The distant metastatic-free survival (DMFS) was analyzed in an institutional series of 1,649 patients who underwent negative SNB (i.e., AJCC pStage I-II melanoma). Older age was associated with significantly shorter DMFS (mean [95% CI]: 15.9 [15.2-16.6] years vs. 18.4 [17.7-19.0] years, log rank p<0.001; FIG. 1D). Moreover, in a multivariate Cox proportional hazards model adjusting for known prognostic factors (e.g., Breslow depth and the presence of ulceration and lymphovascular invasion), older age remained independently associated with a higher risk of distant recurrence (HR 1.49, 95% CI 1.01-2.20) (Table 2).

TABLE 2
Multivariable Cox Regression Model for distant metastatic-free
survival in non-metastatic melanoma patients (n = 1,649)

| Parameter | Comparison | HR* | 95% CI | p-value |
|---|---|---|---|---|
| Age, years | <50 | Ref | Ref | 0.045 |
| | ≥50 | 1.49 | 1.01-2.20 | |
| Ulceration | No | Ref | Ref | <0.001 |
| | Yes | 2.44 | 1.61-3.68 | |
| | No | Ref | Ref | 0.065 |
| Lymphovascular | Yes | 1.79 | 0.97-3.33 | |
| invasion | | | | |

| | | | |
|---|---|---|---|
| Breslow depth, mm (increasing) | 1.16 | 1.10-1.22 | <0.001 |

Next to determine if we could experimentally recapitulate these data, mCherry labeled Yumm1.7 cells, derived from the BrafV600E/Cdkn2a−/−/Pten−/− mouse model of melanoma(17), were injected into the dermis of young (8 weeks) or aged (52 weeks) C57/BL6 mice and tumor burden in the draining inguinal lymph node and the lungs was quantified after 5 weeks. Tumor cells were identified by positive immunhistochemical staining for mCherry, which is specific for the mCherry labeled Yumm1.7 cells. As with the human epidemiologic studies, the aged mice had reduced lymph node metastases but increased tumor burden in the lung (FIG. 1E and FIG. 1F). Together these data confirm that aging increases visceral metastatic dissemination despite reduced lymphatic metastasis.

### HAPLN1 Loss in the Aged Microenvironment Contributes to ECM Changes Leading to Loss of Lymphatic Vessel Integrity.

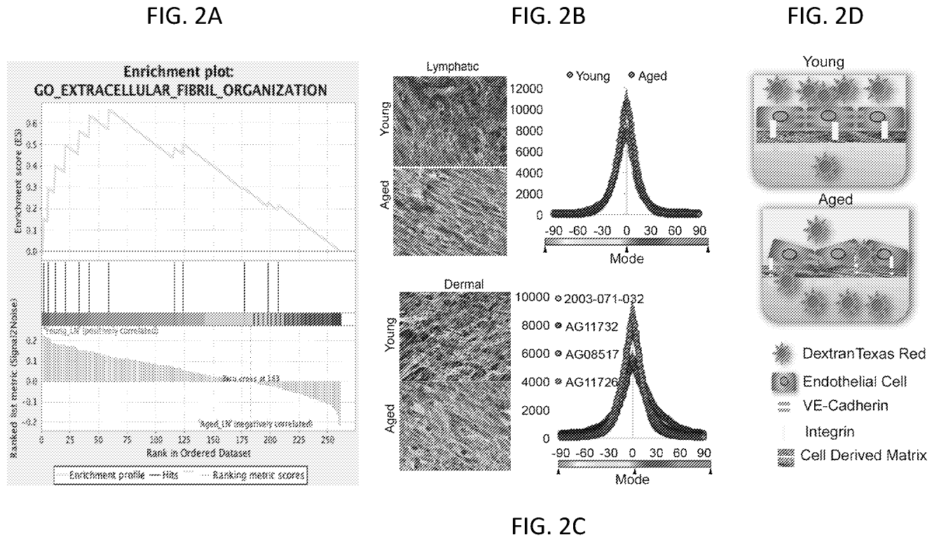

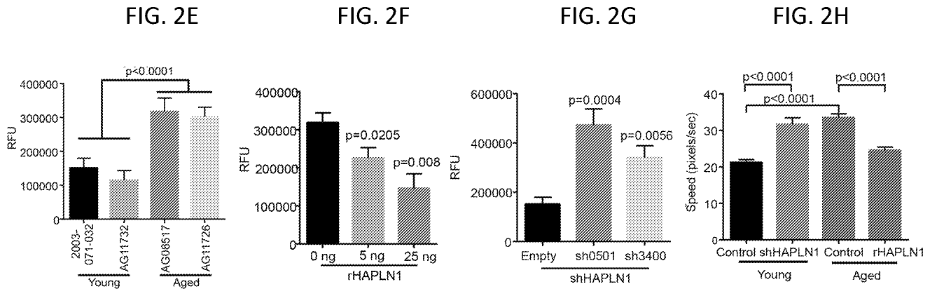

We hypothesized that changes in lymphatic architecture underlie age-related changes in lymphatic permeability, both of the lymphatic vessel and the lymph node itself. Melanoma cells travel via afferent lymphatic vessels to enter the subcapsular sinus of lymph nodes(18). The lymphatic vessels are embedded in fibroblast-secreted ECM, where lymphatic vessel integrity is maintained by the cell-cell contact between the endothelial cells that make up the lymph vessels, as well as the anchoring of these cells to the ECM, which further stabilizes the connection. To test this, lymph node specimens available in the TCGA from young (<50 years) and aged (>50 years) melanoma patients were used to perform GSEA analysis for ECM fibril organization, where the signature showed significant enrichment in young lymph nodes relative to the aged lymph node samples (FIG. 2A, p=0.008). To explore age-related differences in matrix orientation in vitro, lymphatic fibroblasts isolated from young or aged human donors were used to prepare matrices and analyzed for fibronectin fiber orientation, and compared to matrices made by dermal fibroblasts from young and aged human donors. Similar age related decreases in matrix complexity were observed in both dermal and lymphatic (FIG. 2B and FIG. 2C) fibroblast matrices, supporting previous observations of the broad similarities between fibroblasts of these two anatomic sites (19).

In order to determine whether age-dependent structural changes in the ECM impact on endothelial permeability, acellular matrices produced in vitro by young or aged fibroblasts in transwells were reconstituted with an endothelial (HUVEC) monolayer and dextran-conjugated Texas Red fluorescent dye was added to the upper chamber (see schematic in FIG. 2D). HUVEC cells were utilized after initial failures to reliably grow lymphatic endothelial cells in vitro. The integrity of the endothelial cell monolayer was determined with spectrophotometry by measuring the concentration of Texas Red that had diffused into the bottom well after 30 minutes. There was significantly increased endothelial permeability in the context of the aged fibroblast matrix in multiple cell lines (FIG. 2E). These data support the concept that changes in the aged ECM can destabilize lymphatic vessel integrity, leading to increased permeability.

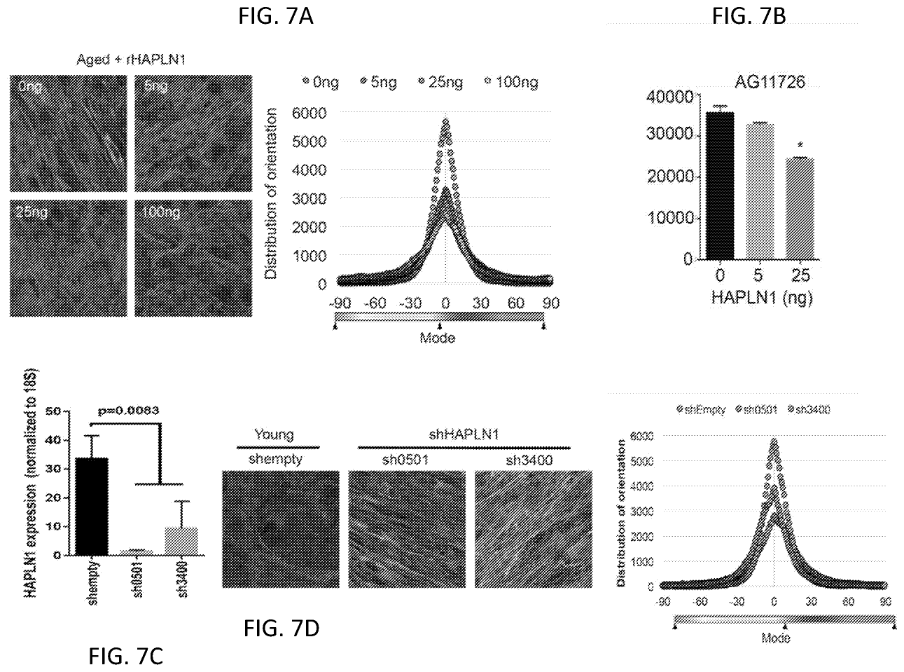

Previous work from our lab has demonstrated an age-dependent loss of fibroblast-secreted HAPLN1 in the skin, which in turn impairs ECM structure and accelerates melanoma invasion (13). To determine whether HAPLN1 could contribute to the observed age-related changes in lymphatic vessel permeability, the transwell permeability assay was performed using an aged fibroblast cell line treated with increasing concentrations of rHAPLN1. The treatment of aged fibroblasts with rHAPLN1 showed increased matrix complexity (FIG. 7A). When produced in transwells and reconstituted with endothelial cells, we observed a step-wise decrease in endothelial permeability (FIG. 2F, second cell line in FIG. 7B). Likewise, endothelial cells plated on an acellular ECM produced by young fibroblasts following HAPLN1 knockdown (FIG. 7C) lost matrix complexity (FIG. 7D) and subsequently evidenced a significant increase in endothelial permeability (FIG. 2G). Next, to see if melanoma cells could more easily transverse the barriers created by aged vs. young fibroblasts, we labeled melanoma cells, plated them atop endothelial cells attached to matrices laid down by young or aged fibroblasts in which HAPLN1 had been manipulated, and then measured the velocity of the tumor cells. In the presence of HAPLN1-containing matrices, tumor cells were less able to cross the endothelial cell barrier (FIG. 2H). These data suggest that HAPLN1 loss during aging, which we have previously shown destabilizes the ECM, can contribute to lymphatic permeability.

### Endothelial Cell-Cell Adhesion Mediates Permeability of Aged Lymphatic Matrix

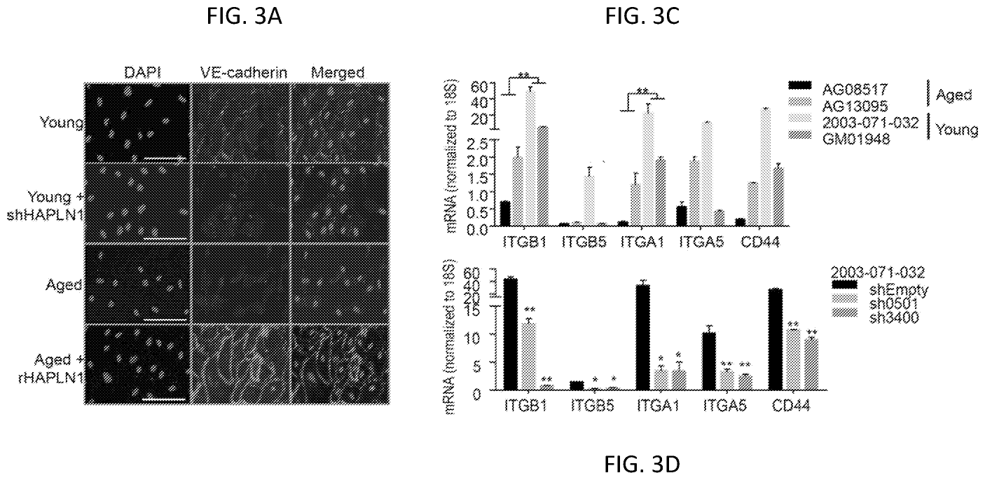

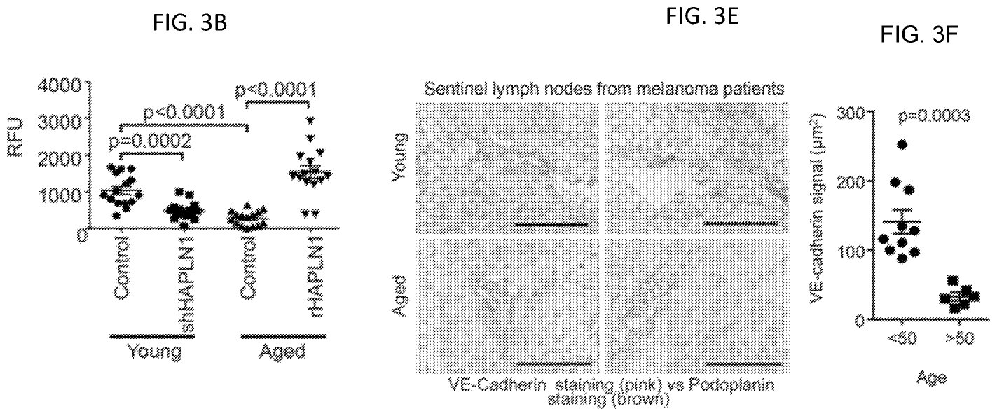

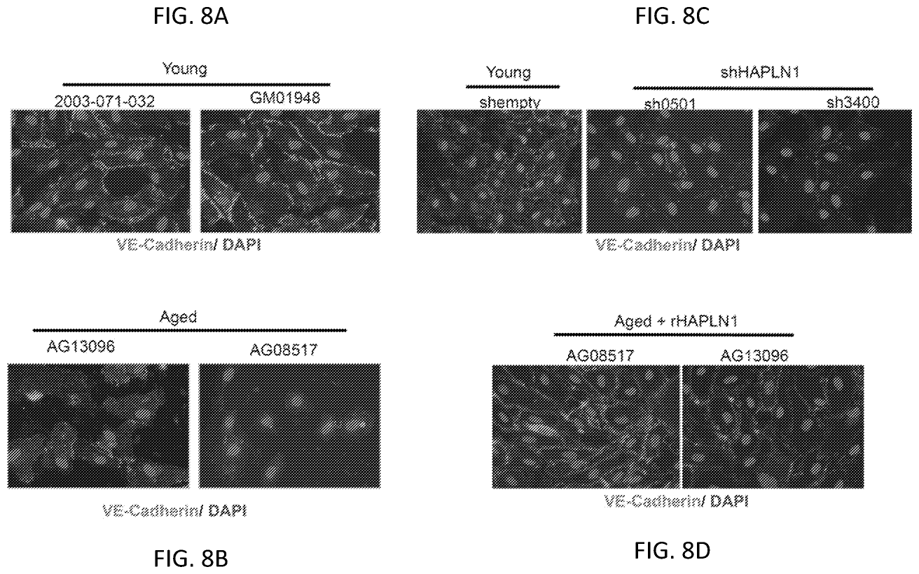

We next assessed whether changes in lymphatic permeability might be explained by differences in cell-cell adhesion due to ECM degradation. VE-cadherin is a critical component of endothelial adherens junctions and mediates vessel permeability (20). Endothelial cells were plated on acellular matrices following extraction of young or aged fibroblasts, and VE-cadherin expression was assessed by immunofluorescence. HUVECs on a young fibroblast matrix evidenced strong VE-cadherin membrane staining with a zipper-like appearance between neighboring cells, which was reduced in endothelial cells plated on an aged fibroblast matrix (FIG. 3A and FIG. 3B; Additional lines, FIG. 8A and FIG. 8B). There was also reduced immunofluorescence signal in the aged context, suggesting such structural changes were related to reduced cell surface protein (FIG. 3A and FIG. 3B). As the knockdown of HAPLN1 in young fibroblasts or the additional of rHAPLN1 to aged fibroblasts was sufficient to change endothelial permeability in a transwell assay, we next investigated whether such changes would alter VE-cadherin signaling. The treatment of fibroblasts from aged donors with rHAPLN rescued HUVEC endothelial adherens junctions, where VE-cadherin signaling was comparable to that observed in the context of young fibroblasts. Additionally, the knockdown of HAPLN1 in young fibroblasts reduced the complexity and overall expression of VE-cadherin in the HUVEC monolayer (FIG. 3A and FIG. 3B; FIG. 8C and FIG. 8D).

Lymphatic endothelial permeability is also dependent on anchorage to the ECM by integrin protein complexes(15). HUVECS were plated on acellular matrices following extraction of multiple young or aged fibroblasts and the relative mRNA expression of integrin subunits (α1, α5, β1, β5) and CD44 was assessed by qPCR. HUVEC expression of α1 and β1 integrins was significantly reduced in the aged matrix relative to the ECM produced by young fibroblasts (FIG. 3C). Moreover, endothelial cells plated onto a matrix produced by two different knockdown shHAPLN1 young fibroblasts had reduced integrin expression (FIG. 3D), supporting a causal role for HAPLN1-mediated ECM complexity and endothelial integrity. To explore the role of endothelial cell adhesion in vivo, human SNB specimens were co-stained for VE-cadherin and podoplanin by immunohistochemistry. Patients provided written informed consent for the procedure, and de-identified samples were obtained under exemption. Podoplanin is a specific lymphatic endothelial glycoprotein that is not expressed in blood vessel endothelium (21). In aged patients, there was a near absence of VE-cadherin staining (pictured in red; FIG. 3E) in the lymphatic channels identified (pictured in brown). In contrast, lymphatic channels in the SNB specimens from young patients evidenced frequent co-localization of podoplanin with VE-cadherin, where channels appear pink, and this is quantitated. These data confirm the age-dependent loss of VE-cadherin in lymphatic endothelium that may underlie observed changes in permeability.

### HAPLN1 Loss During Aging Affects Permeability of the Lymph Node Sinus as Well as the Lymphatic Vasculature.

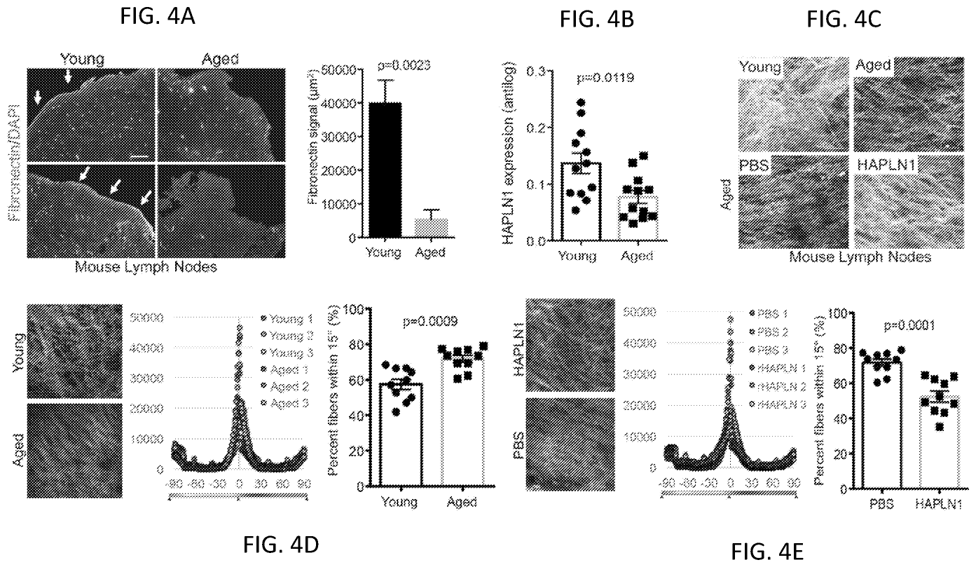

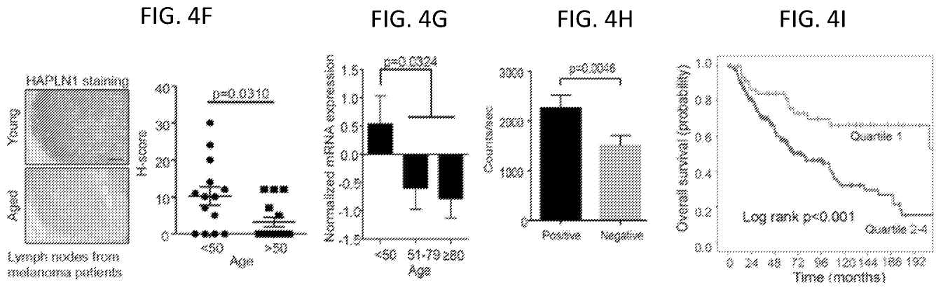

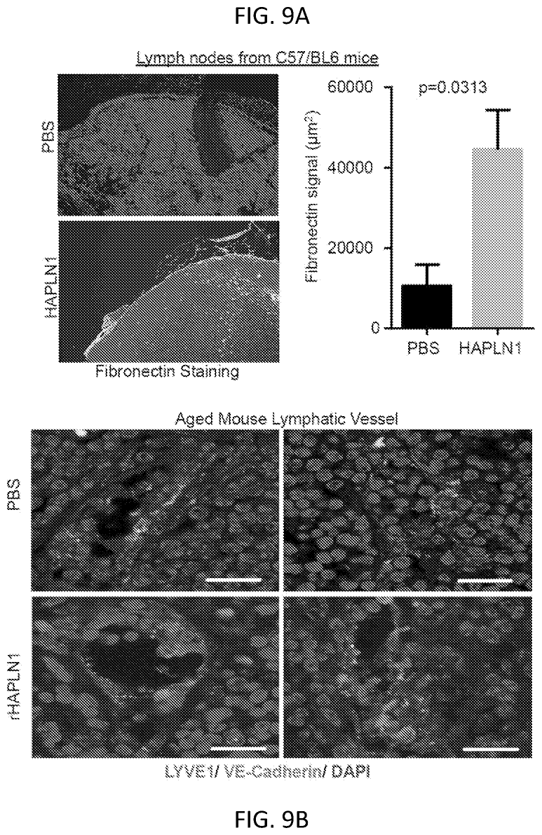

The subcapsular sinus, lined by lymphatic endothelial cells, regulates tumor motility through the lymph node (22). Since the subcapsular sinus is continuous with the endothelium of the afferent lymph vessels, we hypothesized that similar age-related changes would be present in the lymph nodes, and may account for differences in their function to contain metastatic cells and prevent dissemination to visceral sites. Staining of the young and aged mouse lymph nodes for fibronectin demonstrated a loss of fibronectin in the stroma around the lymph node capsule of aged mice (FIG. 4A). We asked whether, as with the previous observations above, this could be due to changes in HAPLN1 levels. HAPLN1 expression was significantly lower in the aged murine lymph nodes (FIG. 4B). To study the impact of HAPLN1 in vivo, inguinal lymph nodes from aged C57/BL6 mice were treated with rHAPLN1 and their collagen architecture was evaluated using two-photon microscopy. Treatment increased the ECM complexity of the lymphatic pericapsular space (FIG. 4C). Using anisotropic/isotropic quantification of fiber alignment, we also saw that loss of matrix complexity with age (FIG. 4D) could be reverted by treatment with HAPLN1 (FIG. 4E, FIG. 9A). This in turn restored VE-cadherin staining in regions of lymphatic endothelium (FIG. 9B). Similarly in patient samples, there was evidence of reduced HAPLN1 in the sentinel lymph nodes of aged melanoma patients, both at the protein (FIG. 4F) and transcriptomic (FIG. 4G) level.

To evaluate the association between HAPLN1 expression and lymphatic permeability in vivo, the Geiger counts corresponding to the sentinel lymph nodes of melanoma patients (n=86) were correlated with HAPLN1 immunohistochemical staining. Patients provided written informed consent for the procedure, and de-identified samples collected under an IRB exemption were used. A total of 21 (23.3%) patients had HAPLN1-positive SLNs. The Geiger counts of HAPLN1 positive SLNs were significantly higher than those without any HAPLN1 staining (median [IQR]: 2364 [1079-3235] counts/sec vs. 2123 [831-1667] counts/sec; p=0.005). Because of the potential bias of age on HAPLN1 expression, the analysis was repeated in the older (age >50 years) patient subset, where a similar relationship between higher HAPLN1 and increased radiotracer retention was observed (1782 [883-2754] counts/sec vs. 772 [260-1967] counts/sec; p=0.034, FIG. 4H)

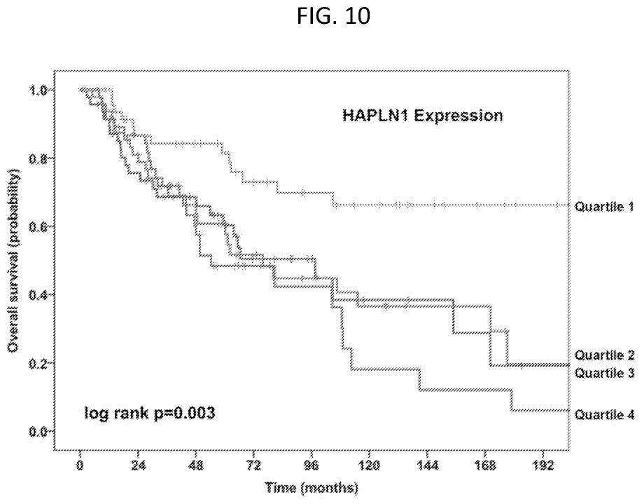

Given these findings, we hypothesized that differences in HAPLN1 expression in the regional lymph nodes of melanoma patients would have prognostic significance for long-term survival. Using a subset of non-metastatic melanoma patients where regional lymphatic tissue was included in the TCGA database (n=192), we observed a threshold effect of improved overall survival associated with the upper quartile of HAPLN1 expression (log rank p<0.001; FIG. 4I), with similar long-term outcomes observed in the lower three quartiles of mRNA expression (FIG. 10). Given potential confounding effects of age on this association, the prognostic value of HAPLN1 expression was confirmed in a multivariate Cox proportional hazards model, which controlled for both AJCC stage and patient age (Table 3). Taken together, these data indicate that loss of HAPLN1 during aging can destabilize the ECM, which in turn can affect the VE-cadherin connections between the lymphatic endothelial cells, and increase permeability of the both the vessels and the nodes.

TABLE 3
Cox proportional hazards model evaluating the impact
of HAPLN1 expression on overall survival, accounting
for pathologic stage and patient age.

| | | | HR* | 95% CI | p-value |
|---|---|---|---|---|---|
| | AJCC | I | Ref | Ref | 0.001 |
| | Pathologic | II | 1.68 | 0.93-3.06 | 0.088 |
| | stage | III | | 1.62-4.53 | <0.001 |
| Age, years (increasing) | | | 1.02 | 1.01-1.04 | 0.002 |
| | HAPLN1 | Q2-4 | Ref | Ref | 0.004 |
| | | Q1 | 0.44 | 0.25-0.77 | |

*Hazard ratio (HR) indicates relative hazard for death and was adjusted for all variables included

### HAPLN1-Dependent Lymph Node Permeability Determines Melanoma Progression

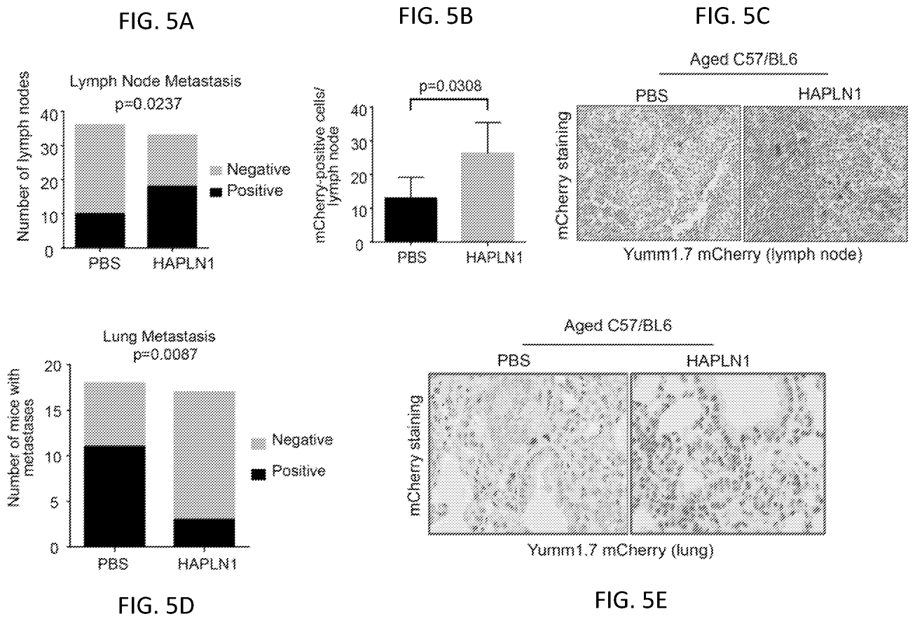

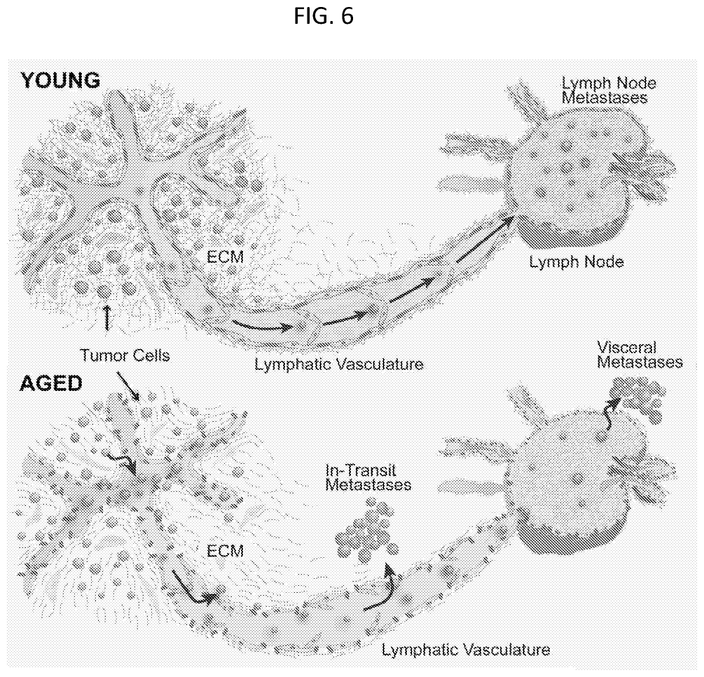

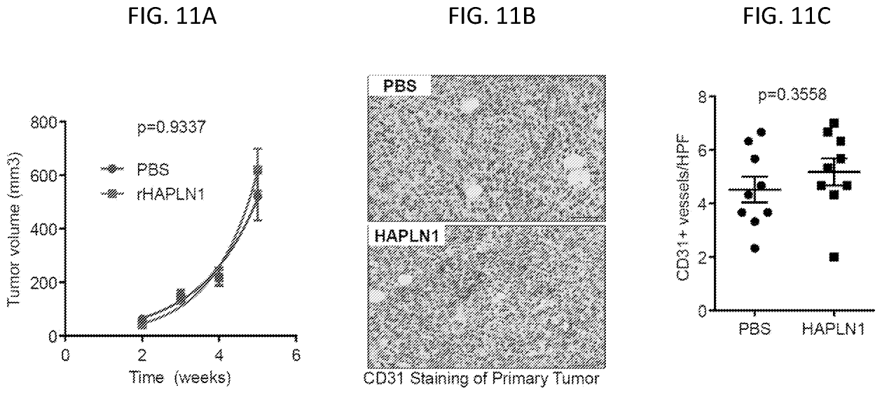

To determine if HAPLN1-mediated permeability would be sufficient to change the patterns of metastasis, the draining lymph nodes of aged C57/BL6 mice were treated with rHAPLN1 or PBS control preceding heterotopic tumor cell injection. In contrast to prior experiments where peritumoral injection of rHAPLN1 into aged mice reduced the size and metastatic potential of primary tumors (13), lymphatic injection of rHAPLN1 (into the draining lymph nodes) had no effect on tumor size (FIG. 11A), which may be expected given its local effects of collagen matrix orientation. We hypothesized that HAPLN1 treatment of aged lymph nodes would decrease lymphatic permeability and thereby decrease the rate of distant metastasis. In support, aged mice treated with rHAPLN1 had greater rates of lymphatic micrometastases (FIG. 5A) as well as greater lymphatic tumor burden, suggesting decreased “escape” from the draining lymph node (FIG. 5B and FIG. 5C). While lymph node metastases are associated with an unfavorable prognosis for melanoma patients, surgical resection of locoregional disease (i.e., the primary site and the draining lymphatic basin) is often an effective treatment not typically available to patients with disease progression to visceral sites. Hence, the containment of tumor metastasis to lymphatic basins may have therapeutic implications. Accordingly, rHAPLN1 treatment of draining lymph nodes in this mouse cohort led to a reduced frequency of distant pulmonary micrometastasis despite higher rates of lymphatic metastasis (FIG. 5D-E). There were no differences in primary tumor angiogenesis that may have provided an alternative (non-lymphatic) pathway for visceral metastatic spread (FIG. 11B and FIG. 11C). Such observations support the hypothesis of sequential progression of melanoma tumor cells through the lymphatic system to visceral sites, and the role of HAPLN1-mediated ECM integrity in regulating lymphatic permeability (summarized in FIG. 6).

### Example 3: Discussion

Melanoma patients of older age experience a higher rate of distant metastasis and inferior overall survival. In particular, the dissemination of melanoma cells beyond the primary site and regional lymphatic basin that are the primary targets for surgical extirpation, presents a clinical dilemma with poor therapeutic options. While the relationship between age and sentinel lymph node positivity has been previously described, herein we identify (1) age-related alterations in the perilymphatic extracellular matrix that mediates lymphatic permeability via destabilization of VE-Cadherin junctions, and (2) a novel role of HAPLN1 in lymphatic ECM integrity, including its prognostic role in human patients and its potential therapeutic value in reducing visceral metastases.

Age-dependent loss of ECM integrity has been demonstrated in studies of skin, and we have recently determined its impact on primary melanoma tumor activity (13). Whether similar age-related degradation occurs in stroma surrounding lymphatic vessels and nodes has rarely been studied. In one previous analysis of mesenteric lymphatic vessels isolated from young and aged rats, aging was associated with a decrease in gap junction proteins and the thickness of the endothelial cell glycocalyx, as well as increased hyperpermeability to bacterial pathogens(15). Our data confirm similar changes in lymphatic endothelial gap junctions and integrins in both in vitro lymph vessel constructs and in vivo lymph node biopsies from melanoma patients and mice. Moreover, age-related changes in lymphatic endothelium were sufficient to mediate permeability in transwell assays using human cell lines as well as in melanoma patients receiving technetium dye for sentinel lymph node detection. Notably, in these studies utilizing mouse and human tissues, the lymph nodes were studied separately from the afferent lymphatic vessels (that transport tumor cells from the primary tumor site in the dermis) due to technical limitations of the models and availability of tissues. Unfortunately, this prohibited the specific study of the effects of aging on dermal lymphatic vessels apart from the draining lymph nodes in vivo. However, given the greater rates of in-transit metastasis that develop in older melanoma patients, we hypothesize that similar changes in permeability occur in the dermal lymphatic vessels (FIG. 6). New techniques will be needed to explore the effects of aging on collecting dermal lymphatics and their role in mediating in-transit disease.

Second, the identification of age-related loss of lymphatic HAPLN1 provided a prognostic biomarker and potential therapeutic target. The loss of HAPLN1 with aging was demonstrated at both the protein and gene expression level. Importantly, patients with high lymphatic HAPLN1 expression were 56% less likely to die, regardless of age and disease stage. The prognostic utility of HAPLN1 as assessed by IHC, which could be more readily incorporated into current clinical practice, remains to be determined. Nevertheless, the targeting of aged lymph nodes with rHAPLN1 in vivo was sufficient to change lymph node architecture and abate the development of visceral metastasis. HAPLN1—and possibly similar ECM-associated proteins—can be targeted to reduce the rates of visceral metastasis. At a minimum, incorporation of HAPLN1 expression, particularly in elderly melanoma patients with negative sentinel lymph node biopsies, into clinical algorithms guiding postoperative surveillance and adjuvant systemic therapy may improve the management of those patients at greatest risk for the development of visceral metastasis.

By inference, these data support the sequential progression model of tumor metastasis—whereby tumor spreads from primary site, to lymph node, and then to distant visceral sites. The sequential cascade model is supported by clinical observation, particularly that the development of lymphatic disease often precedes distant metastasis(23, 24). Yet the lack of survival benefit following lymphadenectomy in melanoma patients has inspired the alternate view that lymphatic and visceral metastasis develop independently(25). However, these two observations can be reconciled upon recognition that lymphadenectomy can only improve survival if performed prior to tumor spread from the lymph nodes to distant sites. In our experiments, young mice or aged mice treated with lymphatic rHAPLN1 had increased lymphatic metastasis and concurrently decreased pulmonary metastasis, providing direct causal support for lymphatic dissemination preceding hematogenous spread. However, the timing of cancer cell trafficking in this cascade is not known, and likely varies by primary tumor burden, disease site and host-related factors. Still, the removal of lymph nodes at an early stage prior to spread beyond the regional basin would likely be curative. Alternatively, strategies to improve lymphatic integrity prior to lymphadenectomy may decrease the false negative rate of sentinel lymph node biopsy and improve long-term survival.

While lymphatic metastasis is the dominant pattern of tumor dissemination in melanoma patients, peritumoral angiogenesis may provide an alternative pathway for tumor egress that bypasses the lymphatic system altogether. Our previous work identified secreted frizzled-related protein 2 (sFRP2) as an age-dependent component of the fibroblast secretome (26), and sFRP2 promotes angiogenesis via activation of the Wnt/Ca2+ signaling pathway (27). Moreover, the targeting of sFRP2 in breast tumor endothelium inhibits tumor angiogenesis and growth(27). In direct support, aged mice have higher expression of sFRP2 than young mice are more likely to develop tumors with a higher density of CD31-positive vessels (26), providing a mechanism by which tumor cells may reach visceral sites independent of the age-related changes in lymphatic permeability. These are not mutually exclusive observations: indeed the aged tumor microenvironment may promote visceral metastasis by multiple mechanisms that lead to inferior clinical outcomes in melanoma patients. Still, the decrease in visceral metastasis simultaneous with the increase in lymphatic metastasis observed in these experiments following the treatment of lymph nodes (and not the primary tumor or peritumoral lymphatics) with rHAPLN1 highlights the causal role for lymph node permeability in mediating melanoma dissemination.

In conclusion, these data suggest that aging leads to degradation of the perilymphatic stroma, which alters lymph node permeability and dictates the route of metastasis. Age should be an important consideration in the management and treatment of melanoma patients, and requires specific strategies to improve outcomes for this growing high-risk patient population.

### Example 4: Administration of HAPLN1

Recombinant HAPLN1 is injected into the draining lymph node of aged patients with melanoma. Dosage ranges from 1 μg to 1 mg.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

### REFERENCES

1. Balch C M, Soong S J, Gershenwald J E, Thompson J F, Reintgen D S, Cascinelli N, et al. Prognostic factors analysis of 17,600 melanoma patients: validation of the American Joint Committee on Cancer melanoma staging system. J Clin Oncol. 2001; 19:3622-34.
2. Amin M B, Greene F L, Edge S B, Compton C C, Gershenwald J E, Brookland R K, et al. The Eighth Edition AJCC Cancer Staging Manual: Continuing to build a bridge from a population-based to a more “personalized” approach to cancer staging. CA Cancer J Clin. 2017; 67:93-9.
3. Balch C M, Soong S J, Gershenwald J E, Thompson J F, Coit D G, Atkins M B, et al. Age as a prognostic factor in patients with localized melanoma and regional metastases. Ann Surg Oncol. 2013; 20:3961-8.
4. Lasithiotakis K, Leiter U, Meier F, Eigentler T, Metzler G, Moehrle M, et al. Age and gender are significant independent predictors of survival in primary cutaneous melanoma. Cancer. 2008; 112:1795-804.
5. Morton D L. Overview and update of the phase III Multicenter Selective Lymphadenectomy Trials (MSLT-I and MSLT-II) in melanoma. Clin Exp Metastasis. 2012; 29:699-706.
6. Wissmann C, Detmar M. Pathways targeting tumor lymphangiogenesis. Clin Cancer Res. 2006; 12:6865-8.
7. Faries M B, Thompson J F, Cochran A J, Andtbacka R H, Mozzillo N, Zager J S, et al. Completion Dissection or Observation for Sentinel-Node Metastasis in Melanoma. N Engl J Med. 2017; 376:2211-22.
8. Page A J, Li A, Hestley A, Murray D, Carlson G W, Delman K A. Increasing Age Is Associated with Worse Prognostic Factors and Increased Distant Recurrences despite Fewer Sentinel Lymph Node Positives in Melanoma. Int J Surg Oncol. 2012; 2012:456987.
9. Chao C, Martin R C, 2nd, Ross M I, Reintgen D S, Edwards M J, Noyes R D, et al. Correlation between prognostic factors and increasing age in melanoma. Ann Surg Oncol. 2004; 11:259-64.
10. Kang J S, Kawakami Y, Bekku Y, Ninomiya Y, Izpisua Belmonte J C, Oohashi T. Molecular cloning and developmental expression of a hyaluronan and proteoglycan link protein gene, crtl1/hapln1, in zebrafish. Zoological science. 2008; 25:912-8.
11. Huang-Lee L L, Nimni M E. Crosslinked CNBr-activated hyaluronan-collagen matrices: effects on fibroblast contraction. Matrix Biol. 1994; 14:147-57.
12. Karjalainen J M, Tammi R H, Tammi M I, Eskelinen M J, Agren U M, Parkkinen J J, et al. Reduced level of CD44 and hyaluronan associated with unfavorable prognosis in clinical stage I cutaneous melanoma. Am J Pathol. 2000; 157:957-65.
13. Kaur A, Ecker, B. L., Douglass, S. M., Kugel C. H., 3rd, Webster, M. R., Almeida F. V., et al. Remodeling of the collagen matrix in aging skin promotes melanoma metastasis and affects immune cell motility. Cancer Discovery. 2018; In Press.
14. Roozendaal R, Mebius R E, Kraal G. The conduit system of the lymph node. Int Immunol. 2008; 20:1483-7.
15. Zolla V, Nizamutdinova I T, Scharf B, Clement C C, Maejima D, Akl T, et al. Aging related anatomical and biochemical changes in lymphatic collectors impair lymph transport, fluid homeostasis, and pathogen clearance. Aging Cell. 2015; 14:582-94.
16. Karaman S, Buschle D, Luciani P, Leroux J C, Detmar M, Proulx S T. Decline of lymphatic vessel density and function in murine skin during aging. Angiogenesis. 2015; 18:489-98.
17. Dankort D, Curley D P, Cartlidge R A, Nelson B, Karnezis A N, Damsky W E, Jr., et al. Braf(V600E) cooperates with Pten loss to induce metastatic melanoma. Nat Genet. 2009; 41:544-52.
18. Nathanson S D. Insights into the mechanisms of lymph node metastasis. Cancer. 2003; 98:413-23.
19. Vega F, Coombes K R, Thomazy V A, Patel K, Lang W, Jones D. Tissue-specific function of lymph node fibroblastic reticulum cells. Pathobiology. 2006; 73:71-81.
20. Giannotta M, Trani M, Dejana E. VE-cadherin and endothelial adherens junctions: active guardians of vascular integrity. Dev Cell. 2013; 26:441-54.
21. Baluk P, McDonald D M. Markers for microscopic imaging of lymphangiogenesis and angiogenesis. Ann N Y Acad Sci. 2008; 1131:1-12.
22. Podgrabinska S, Skobe M. Role of lymphatic vasculature in regional and distant metastases. Microvasc Res. 2014; 95:46-52.
23. Patel J K, Didolkar M S, Pickren J W, Moore R H. Metastatic pattern of malignant melanoma. A study of 216 autopsy cases. Am J Surg. 1978; 135:807-10.
24. Leong S P, Cady B, Jablons D M, Garcia-Aguilar J, Reintgen D, Jakub J, et al. Clinical patterns of metastasis. Cancer Metastasis Rev. 2006; 25:221-32.
25. Morton D L, Thompson J F, Cochran A J, Mozzillo N, Nieweg O E, Roses D F, et al. Final trial report of sentinel-node biopsy versus nodal observation in melanoma. N Engl J Med. 2014; 370:599-609.
26. Kaur A, Webster M R, Marchbank K, Behera R, Ndoye A, Kugel C H, 3rd, et al. sFRP2 in the aged microenvironment drives melanoma metastasis and therapy resistance. Nature. 2016; 532:250-4.
27. Siamakpour-Reihani S, Caster J, Bandhu Nepal D, Courtwright A, Hilliard E, Usary J, et al. The role of calcineurin/NFAT in SFRP2 induced angiogenesis—a rationale for breast cancer treatment with the calcineurin inhibitor tacrolimus. PLoS One. 2011; 6:e20412.
28. Franco-Barraza J, Beacham D A, Amatangelo M D, Cukierman E. Preparation of Extracellular Matrices Produced by Cultured and Primary Fibroblasts. Curr Protoc Cell Biol. 2016; 71:10 9 1-9 34.
29. Berencsi K, Meropol N J, Hoffman J P, Sigurdson E, Giles L, Rani P, et al. Colon carcinoma cells induce CXCL11-dependent migration of CXCR3-expressing cytotoxic T lymphocytes in organotypic culture. Cancer Immunol Immunother. 2007; 56:359-70.
30. Cancer Genome Atlas N. Genomic Classification of Cutaneous Melanoma. Cell. 2015; 161:1681-96.

All publications cited in this specification priority document U.S. Provisional Patent Application No. 62/740,330, filed Oct. 2, 2018, are incorporated herein by reference. While the invention has been described with reference to particular embodiments, it will be appreciated that modifications can be made without departing from the spirit of the invention. Such modifications are intended to fall within the scope of the appended claims.

1. A method of preventing or decreasing the risk of cancer metastasis, comprising upregulating or delivering HAPLN1 to a subject in need thereof.

2-3. (canceled)

4. The method according to claim 1, comprising delivering rHAPLN1.

5. The method according to claim 4, wherein the rHAPLN1 is delivered to a draining lymph node.

6. The method according to claim 4, wherein the rHAPLN1 is delivered to a tumor site.

7. The method according to claim 1, comprising delivering a vector which encodes HAPLN1.

8. The method according to claim 4, wherein the vector is delivered to a lymph node or lymphatic vessel.

9. The method according to claim 4, wherein the vector is delivered to a tumor site.

10. The method according to claim 7, wherein the vector is a viral vector.

11. The method according to claim 1, wherein the subject is an older adult.

12. A method of predicting likelihood of survival in a subject having cancer, comprising assaying for HAPLN1 protein or RNA expression in lymphatic tissue.

13. The method of claim 12, wherein a higher HAPLN1 level is indicative of a higher chance of survival.

14. A method of predicting likelihood of metastasis in a subject having cancer, comprising assaying for HAPLN1 expression in lymphatic tissue.

15. The method of claim 14, wherein a higher HAPLN1 level is indicative of a lower chance of metastasis.

16. The method according to claim 1, wherein the subject has cancer selected from melanoma, prostate, clear cell renal cell carcinoma, breast cancer, other skin cancers, and any other cancers that can metastasize via the lymphatic system, including but not limited to lung, non-small cell lung, pancreatic, colorectal, head and neck, cervical, endometrial, testicular, and ovarian cancer.

17. The method according to claim 16, wherein the cancer is melanoma.

18. The method according to claim 1, comprising upregulating or delivering HAPLN1 to the ovaries.
